(19) United States
(12) Patent Application Publication
(10) Pub. No.: US 2003/0171573 A1
(43) Pub. Date: Sep. 11, 2003

(54) Multivalently interactive molecular assembly, capturing agent, drug carrier, calcium chelating agent, and drug enhancer

(76) Inventors: Nobuhiko Yui, Ishikawa (JP); Atsushi Maruyama, Kanagawa (JP); Tooru Ooya, Ishikawa (JP)

Correspondence Address: ARMSTRONG,WESTERMAN & HATTORI, LLP, 1725 K STREET, NW, SUITE 1000, WASHINGTON, DC, 20006, US

(21) Appl. No.: 10/230,394

(22) Filed: Aug. 29, 2002

(30) Foreign Application Priority Data
Feb. 27, 2002 (JP) 2002-052474

(51) Int. Cl. A61K 31/724; C08B 37/16

(52) U.S. Cl. 536/103; 514/54

(57) ABSTRACT

A multivalently interactive molecular assembly having a plurality of functional groups or ligands, in which a ratio between Rₕ and R_{g} expressed as Rₕ/R_{g} is 1.0 or less. Here, Rₕ is a hydrodynamic radius calculated from dynamic light scattering (DLS) assay performed in aqueous solution; and R_{g} is a radius of gyration determined based on the Zimm plot generated using data obtained by static light scattering (SLS) assay.

### BACKGROUND OF THE INVENTION

1. Field of the Invention

The present invention relates to a multivalently interactive molecular assembly which can effectively and stably bind to a target substance in vivo or in vitro, a capturing agent comprising said multivalently interactive molecular assembly for capturing an object of interest in vivo or in vitro, a drug carrier which aids administration of a drug, a calcium chelating agent which can effectively chelate calcium, and a drug enhancer which can be administered with a drug to assist in, for example, absorption of the drug.

2. Description of the Related Art

Currently, compounds which comprise ligands having with high affinity for a variety of receptors in vivo are of great interest as novel medicaments since those can affect various functions of those receptors. To obtain such a compound comprising ligands having high affinity for receptors, researchers have made intense studies to develop a variety of such low-molecular weight compounds as well as high-molecular weight compounds containing a great number of ligands which can interact multivalently. However, the conventional low-molecular weight compounds or the water-soluble high molecular weight compounds that comprise any ligands interactive with receptors had limited binding stability and efficiency and thus could not exhibit sufficient interaction multivalency. Particularly, the low-molecular weight compounds had insufficient binding stability since only the limited number of ligands could be incorporated therein. The conventional water-soluble high-molecular weight compounds could have many ligands incorporated therein. However, such conventional high-molecular weight compounds containing many ligands could not be expected to bind effectively and stably to the target receptors. This is because such a high-molecular weight compounds may have a great flexibility and this property tends to associate with each other via hydrophobic interaction of the ligands in the conjugates which leads to form inter- and intra-molecular aggregation having the water-soluble high-polymer molecule as its outer shell. Moreover, both of those low- and high-molecular weight compounds are not degradable, and it was thus impossible to control their binding strength to their targets. Therefore, one has needed the development of a compound comprising ligands having high binding stability (especially those having a binding stability that is controllable in terms of space and time).

Resins comprising repeated acrylic acid units (carbomer and polycarbophil) are known to be useful as drug enhancers to increase transmucosal-permeabilityof protein- or peptide-type drugs. Polyacrylate resins chelate calcium and thereby open the tight-junction of small intestine epithelium. Further, the polyacrylate resins chelate calcium from proteases such as trypsin or chymotrypsin, thereby inhibiting decomposition of protein in an intestinal lumen. As described above, a method for chelating calcium using polyacrylic acid may inhibit the decomposition of a protein and facilitate the permeation of the protein through gastrointestinal tract. However, it has also been reported that the direct binding between polyacrylic acid and enzyme may be the key factor in inhibition of protease activity. This report suggests that an intermolecular bond (e.g., hydrogen bond or electrostatic interaction) via carboxyl group rather than calcium chelation may play an important role in the biological activities. Although polyacrylic acid has useful properties such as calcium chelating ability and non-specific interaction, they were disadvantageous in that these properties can not be controlled. Therefore, it has been needed to develop novel materials of which chelating ability and physical interaction with biological component(s) are controllable and binding to mucosa, drug permeability and protease inhibition can be regulated.

### SUMMARY OF THE INVENTION

The present invention aims at solving the above-described problems in the prior art and attaining the object described below. In summary, the object of the present invention is to provide a multivalently interactive molecular assembly which can effectively and stably bind to a target substance in vivo or in vitro, a capturing agent comprising said multivalently interactive molecular assembly for capturing an object of interest in vivo or in vitro, a drug carrier which aids administration of a drug, a calcium chelating agent which can effectively chelate calcium, and a drug enhancer that can be administered with a drug to assist in, for example, the absorption of the drug.

The present inventors found that a compound with a small flexibility (e.g., a multivalently interactive molecular assembly comprising a plurality of cyclic molecules, a linear molecule that is threaded through the cyclic molecules to hold them together, and capping bulky substituents at the both ends of the linear molecule) did not intramolecularly associate in aqueous conditions even when a great number of functional groups or ligands have been incorporated therein. Also the compound could effectively and stably bind to its target substance(s), and the binding stability of such a compound could be controlled by regulating the amount of the functional groups and/or ligands to be incorporated therein. They also found that, when desired, biodegradable groups can be used as said bulky substituents to reduce the binding multivalency since the in vivo decomposition of the biodegradable groups may lead to the destruction of the entire supramolecular backbone itself, whereby the binding stability of the compound to its target substance(s) is controllable in terms of time and space.

The present inventors also found that polyrotaxane containing, as functional group, carboxyl group incorporated therein can chelate calcium and thus inhibit trypsin activity.

The present invention was developed based on these findings. Hereinafter, means for solving the above-described problems will be described.

In summary, a first aspect of the present invention provides a multivalently interactive molecular assembly comprising a plurality of functional groups and/or ligands, characterized by that Rₕ/R_{g}, which is the ratio between hydrodynamic radius (Rₕ) calculated from dynamic light scattering (DLS) assay performed in aqueous solution and radius of gyration (R_{g}) determined based on the Zimm plot generated using data obtained by static light scattering (SLS) assay, is equal or lower than 1.0.

The ratio (Rₕ/R_{g}) may preferably be from 0.20 to 0.60.

A second aspect of the present invention provides a multivalently interactive molecular assembly comprising a plurality of functional groups and/or ligands, characterized by that the diffusion constant (D) value calculated from the DLS assay performed in aqueous solution may increase as the scattering vector constant (K) increases.

A third aspect of the present invention provides a multivalently interactive molecular assembly comprising a plurality of cyclic molecules, a linear molecule which is threaded through the cyclic molecules to hold them together, and capping bulky substituents at the both ends of the linear molecule, characterized by that at least tow of said a plurality of cyclic molecules are substituted with the functional group and/or the ligand.

Preferably, the bulky substituents can be introduced to the linear molecule via biodegradable linkages thus cleaved from the latter.

The elution time of the multivalently interactive molecular assembly according to the present invention in gel permeation chromatography at a flow rate of 1 ml/min or less may be 1 to 30 minutes shorter than that of any of the cyclic molecules, linear molecules and bulky substituents.

Preferable compounds of multivalently interactive molecular assembly according to the present invention are polyrotaxanes.

The cyclic molecules may preferably be cyclodextrins.

On spectra of one-dimensional ¹H-NMR spectroscopy, glucose C3 and C5 protons present in the cavity of the cyclodextrins may preferably exhibit a 0.1 to 1.0 ppm upfield or downfield shift when compared to those present in the cavity of free cyclodextrin.

The linear molecule threading through the cyclodextrin cavities may preferably exhibit a 0.01 to 1.0 ppm upfield or downfield shift when compared to the linear molecule that is not threading through the cyclodextrin cavities as determined by one-dimensional ¹H-NMR spectroscopy.

Preferably, as determined by a two-dimensional ¹H-NMR spectrum, a cross peak caused by the nuclear Overhauser effect between glucose C3 and C5 protons present in the cavity of the cyclodextrin and protons present in the linear molecule may be detected, and those chemical shifts may be 3.0 to 4.0 ppm for the C3 and C5 protons and 1.0 to 6.0 ppm for the linear molecule respectively.

Preferably, there is no detectable melting peak of the linear molecule in the DSC chart of differential scanning calorimetry.

The functional group may preferably contain a caboxyl group at an end thereof.

Preferable example of functional group containing a caboxyl group at an end thereof may be carboxyalkoxycarbonyl group.

The ligand may be sugar ligand.

A fourth aspect of the present invention provides a multivalently interactive molecular assembly in which a plurality of cyclodextrin molecules are threaded through a linear molecule capped with bulky substituents, characterized by that, in at least two of the cyclodextrin molecules, C6 primary hydroxyl group, C2 secondary hydroxyl group and C3 secondary hydroxyl group each have a peak area which is reduced by 10 to 95% compared to that of the corresponding hydroxyl group in a cyclodextrin with no substituent as determined by two-dimensional ¹H-NMR spectroscopy.

Multivalently interactive molecular assemblies according to the present invention may preferably be used as a capturing agent which can capture an object of interest.

A multivalently interactive molecular assembly according to the present invention may preferably be used as a drug carrier.

A multivalently interactive molecular assembly according to the present invention may preferably be used as a calcium chelating agent.

Alternatively, a multivalently interactive molecular assembly according to the present invention may preferably be used as a drug enhancer.

A capturing agent according to the present invention can capture an object of interest and comprises at least the above-described multivalently interactive molecular assembly according to the present invention.

A drug carrier according to the present invention can be bound to a drug and comprises at least the above-described multivalently interactive molecular assembly according to the present invention.

A calcium chelating agent according to the present invention can chelate calcium and comprises at least the above-described multivalently interactive molecular assembly according to the present invention which contains a functional group having a caboxyl group at an end thereof.

A drug enhancer according to the present invention can be used to enhance the efficacy of the drug and comprises at least the above-described multivalently interactive molecular assembly according to the present invention that contains a functional group having a caboxyl group at an end thereof.

Polyrotaxane according to the present invention may be used in the above-described multivalently interactive molecular assembly according to the present invention.

### BRIEF DESCRIPTION OF THE DRAWINGS

FIGS. 1A through 1E show the results obtained by gel permeation chromatography (GPC).

FIGS. 2A through 2C show the results obtained by 750 MHz ¹H-NMR spectroscopy.

FIG. 3 shows a schematic view of a biotin-polyrotaxane conjugate and a streptavidin-immobilized surface illustrating the binding of the two.

FIG. 4 shows SPR-curves illustrating the binding of biotin-polyrotaxane conjugate to the streptavidin-immobilized surface.

FIG. 5 shows SPR-curves illustrating the binding/dissociation of biotin-polyrotaxane conjugate.

FIG. 6 shows linear plots illustrating dissociation constant between streptavidin and biotin-polyrotaxane conjugate determined from the dissociation curves in FIG. 3.

FIGS. 7A and 7B show inhibition curves illustrating the binding inhibition of streptavidin to the biotin-immobilized sensor surface by the biotin molecule in the conjugate.

FIG. 8 shows the relationship between the fractional inhibition and conjugate concentration.

FIGS. 9A and 9B show conceptual views of binding.

FIG. 10 shows the results obtained by ¹H-NMR analysis of 132CEE-α/E4-PHE-Z.

FIG. 11 shows the results obtained by GPC of 132CEE-α/E4-PHE-Z and 6CEE-α-CD.

FIG. 12 shows the solubility of 132CEE-α/E4-PHE-Z and 6CEE-α-CD in PBS at different pH conditions.

FIG. 13 shows the results obtained by calcium binding assay.

FIG. 14 shows the effects of various compounds including “CEE-Polyrotaxane” on trypsin activity.

FIG. 15 shows trypsin inhibition factors (IFs).

FIG. 16 shows the effects of the length of poly(ethylene glycol) chain on trypsin inhibition.

FIG. 17 shows the IF values (representing trypsin inhibition) for CEE-polyrotaxanes with different number of α-CDs.

FIG. 18 shows a diagram illustrating the inhibition of hemagglutination by various Mal-polyrotaxane conjugates.

FIG. 19 shows the relationship between the threading ratio α-CD and the inhibitory effect.

### DESCRIPTION OF THE PREFERRED EMBODIMENTS

The first aspect of a multivalently interactive molecular assembly according to the present invention may comprise a plurality of functional group(s) and/or ligand(s), and be characterized by that (Rₕ/R_{g}), the ratio between radius of gyration (R_{g}) calculated based on the Zimm plot generated using data obtained by static light scattering (SLS) assay and a hydrodynamic radius (Rₕ) calculated from dynamic light scattering (DLS) assay performed in aqueous solution, is equal or lower than 1.0. The ratio (Rₕ/R_{g}) may preferably be from 0.20 to 0.60.

Conventional multivalently interactive molecular assembly such as spherical micelles, liposomes and particles had a ratio (Rₕ/R_{g}) of 1.28 to 1.30. Conventional polymeric multivalently interactive molecular assemblies generally take a spherical form (which is energy-stable) and may thus intramolecularlly associated with each other when many functional groups and/or ligands have been incorporated therein. Therefore, only the limited number of functional groups and/or ligands are available for association with their target(s), which resulted in low binding stability. On the contrary, a multivalently interactive molecular assembly according to the present invention having a small flexibility may have a small intramolecular association and can therefore bind effectively and stably to the target substance(s) even when many functional groups and/or ligands have been incorporated therein.

Dynamic light scattering (DLS) and static light scattering (SLS) can be determined using a light scattering analyzer DLS7000 (available from Otsuka Electronics Co., Ltd.) with a He—Ne laser (at 630 nm, 10 mW) for static light scattering or an Ar laser (at 488 nm, 75 mW) for dynamic light scattering as a light source. Hydrodynamic radius (Rₕ) and radius of gyration (R_{g}) can be calculated by any known methods.

Any atom or atomic group can be used as the above-described functional groups which may be involved in reaction characteristic to the above-described multivalently interactive molecular assembly, and can be suitably selected depending on a particular purpose. Examples of such functional groups include any heteroatoms except for carbon and hydrogen, atomic groups containing any one or more of these heteroatoms, and structures containing multiple bond(s) between carbon atoms. Particular examples include, for example, hydroxyl, alkoxy (such as methoxy, n-butoxy, n-octyloxy, methoxyethoxy or benzyloxy), alkenyloxy, alkynyloxy, aryloxy (such as phenoxy, p-tolyloxy, 4-methoxyphenoxy or 4-t-butylphenoxy), formyl, keto, acyl, aroyl, carboxyl, alkoxycarbonyl (such as methoxycarbonyl, n-butoxycarbonyl or 2-ethylhexyloxycarbonyl), alkenyloxycarbonyl, alkynyloxy carbonyl, aryloxy carbonyl, alkylsulfonyl (such as n-butylsulfonyl or n-dodecylsulfonyl), arylsulfonyl (such as p-tolylsulfonyl, p-dodecylphenylsulfonyl or p-hexadecyloxyphenylsulfonyl), aminoacyl, amino, cyano, imidoyl, mercapto, nitro and sulfone groups, halogen atoms, sulfide-bond-containing groups, disulfide-bond-containing groups, C═C bond-containing groups, C≡C bond-containing groups, and carboxylic anhydride residue, imide residue (such as succinimide ester) and the like. Such the functional groups may also include activated groups such as N-acylimidazole, succinimide ester, p-nitrophenyl ester, pentafluorophenyl ester, methyl ester, tosyl, aldehyde, allyl, methacryl, acryl, halogenated alkyl, isocyanate and thiol groups. These groups may be substituted with any of the aforementioned groups.

In all the functional groups, amino group that may be substituted, carboxyl group that may be substituted, hydroxyl group or any groups that have been substituted with any of these groups are preferable.

There is no limitation for using any ligand if they can specifically bind to its receptor in vivo or in vitro, and can be suitably selected depending on a particular purpose. The terms “ligand” and “receptor” are conceptually used in relation to each other. Therefore, ligand and receptor should not be considered separately but in combination of two materials that can bind to each other. Examples of ligands or receptors include peptides, saccharides, glycoproteins, lipids, glycolipids, nucleic acids, amino acids, low-molecular weight compounds and ions. Combination of ligand and receptor may include any combination of those substances, including: peptide and peptide; peptide and saccharide; saccharide and peptide; saccharide and nucleic acid; and so on. Unlimiting examples of such combination will be listed in Table 1.

TABLE 1

| Ligand | Receptor |
|---|---|
| Tyroxine-phosphorylated | SH2 domain, PTB domain |
| polypeptide | |
| GTP-binding protein which is | Rho GDI |
| associated with GDP which can | |
| be substituted by GTP via | |
| guanine nucleotide exchanger | |
| (e.g., Rh) | |
| GTP-binding protein which is | Target molecule (e.g., Raf serine |
| associated with GTP which can | threonine kinase for Ras |
| be converted into GDP by GTP | |
| hydrolase (e.g., Ras) | |
| Growth factor, cytokine | Growth factor receptor, |
| | cytokine receptor |
| Antigen | Antibody |
| Low molecular weight | Protein kinase C, |
| metabolite, second messenger | cAMP-dependent kinase, |
| or ion | calmodulin |
| ZSugar ligand such as glucose, | Asialoglycoprotein receptor |
| mannose and maltose | |
| Sialic group | Sialic acid receptor |

These functional groups or ligands may bind to the cyclic compound threaded onto the linear compound, directly or via another functional groups.

The second aspect of a multivalently interactive molecular assembly according to the present invention may comprise a plurality of functional group(s) and/or ligand(s), and be characterized by that the diffusion constant (D) value calculated from dynamic light scattering assay performed in aqueous solution may increase as the scattering vector constant (K) value increases. On the contrary, conventional spherical micelles, liposomes or particles have a consistent (D) value regardless of the (K) value.

The third aspect of a multivalently interactive molecular assembly according to the present invention may comprise a plurality of cyclic molecules, a linear molecule that is threaded through the cyclic molecules to hold them together, and capping bulky substituents at the both ends of the linear molecule, and be characterized by that at least two of said a plurality of cyclic molecules are substituted with a functional groups and/or ligands.

This structure may have a property of small flexibility and accompanying advantageous properties, and allow the many cyclic molecules threaded onto the linear molecule to slide along and rotate around the linear molecule, which facilitates target capturing.

Any linear molecules that can be threaded through a plurality of cyclic molecules to hold them together may be used, including hydrophilic or hydrophobic polymers such as polyethylene glycol (PEG), polypropylene glycol (PPG), block random copolymers thereof, poly(amino acids), polysaccharides and fatty acids. Particularly, PEG may be preferably used as a liner molecule since it can be capped with bulky substituents easily.

Preferably the bulky substituents are enough to cap the both ends of the linear molecule to arrest said a plurality of cyclic molecules, including amino acid, oligopeptide, monosaccharide, oligosaccharide, nucleic acid and fluorescent molecule. Particular but unlimiting examples include: oligopeptide comprising repeated unit of any one or more selected from the group consisting of N-benzyloxycarbonyl-L-phenylalanine, alanine, valine, leucine, isoleucine, methionine, proline, phenylalanine, tryptophan, asparatic acid, glutamic acid, glycine, serine, threonine, tyrosine, cysteine, lysine, arginine and histidine, or derivative thereof.

Bulky substituents may preferably be linked to the linear molecule via biodegradable linkages so that the former can be degraded in vivo and thus cleaved from the latter. When the bulky substituents are attached to the both ends of polyrotaxane via a linkage that can be enzymatically or non-enzymatically hydrolyzed (e.g., peptide, amide, ester or phosphodiester bond), hydrolysis of the linkage may release cyclodextrin to the medium over a certain period of time such as from minute to months. In this case, hydrolysis time can be set at on the order of from minute to months. The hydrolysis can be analyzed by, for example, GPC, reverse-phase chromatography or NMR.

In terms of introduction of such bulky biodegradable groups, conventional multivalent binding polymer compound had an disadvantage that the biodegradation of the substituents will be prevented or hindered since enzyme can hardly access to the substituents due to steric hindrance caused by hydrophobic interactions formed in the molecule while the inventive multivalently interactive molecular assembly has an advantage that enzymes can easily access to the ends of the linear molecule to cleave the substituents therefrom due to the small folding and association tendencies of the molecule.

There are no limitation of cyclic molecules if they have at least one of functional groups and/or ligands, including, for example, cyclodextrin (CD), crown ether and cyclofructan. In these molecules, cyclodextrin is preferable. Examples of cyclodextrin include α-, β- and γ-cyclodextrins with different number of glucose unit.

Functional groups and/or ligands can be introduced into cyclodextrin via the hydroxyl group in the cyclodextrin. Such functional groups or ligands may be linked to the hydroxyl group directly or via another functional groups. For example, in the biotin-containing multivalently interactive molecular assembly shown in Structural Example 1 below, the biotin (i.e., ligand) may be introduced into the assembly by linking the hydrazide group in biotin hydrazide to the hydroxyl group of the cyclodextrin via a carbamoyl bond derived from N,N′-carbonyldiimidazole (CDI). Alternatively, 2-aminoethanol may be linked to the cyclodextrin via the carbamoyl bond.

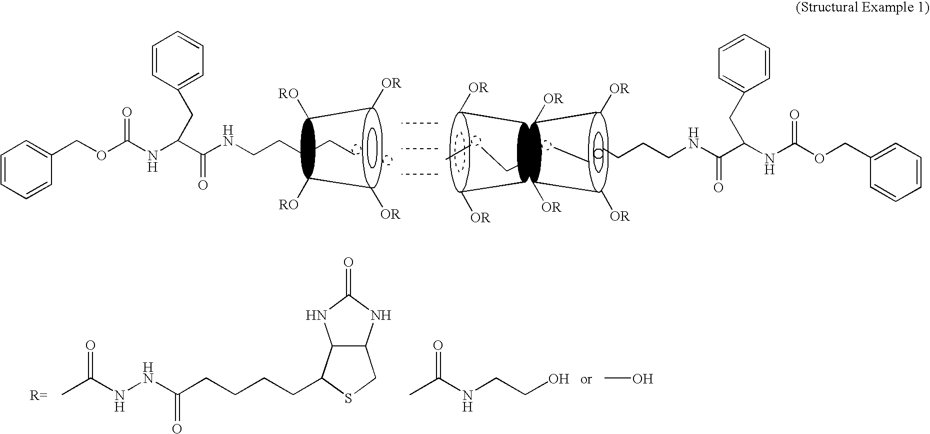

On spectra of one-dimensional ¹H-NMR spectroscopy, glucose C3 and C5 protons present in the cavity of cyclodextrin may preferably exhibit a 0.1 to 1.0 ppm upfield or downfield shift when compared to those present in the cavity of free cyclodextrin. The linear molecule threading through the cyclodextrins may preferably exhibit an upfield or downfield shift when compared to a linear molecule which is not threading through cyclodextrins as determined by the one-dimensional ¹H-NMR spectroscopy.

All the peaks derived from the multivalently interactive molecular assembly in which the polymeric chain is threaded through the cyclodextrin cavity may be approximately 0.01 to 0.5 ppm broader than those derived from the one in which the polymeric chain is not threading through the cyclodextrin cavity.

In two-dimensional ¹H-NMR spectrum, a cross peak caused by the nuclear Overhauser effect between glucose C3 and C5 protons present in the cavity of the cyclodextrin and protons present in the linear molecule. Its chemical shifts were within the range of 3.5 to 4.0 ppm where C3 and C5 protons were observed and 1.0 to 6.0 ppm where the linear molecule was detected.

According to the DSC chart of differential scanning calorimetry (DSC) assay, no melting peak of the linear molecule was detected in the multivalently interactive molecular assembly comprising cyclodextrins and a linear molecule threaded therethrough while a melting peak of the linear molecule was observed in a mixture of cyclodextrins and polymer chain. A multivalently interactive molecular assembly comprising a PEG or PEG copolymer chain as the linear molecule may have a melting temperature of 0 to 200° C.

Multivalently interactive molecular assembly may preferably be polyrotaxane.

The elution time of the multivalently interactive molecular assembly may preferably be 1 to 30 minutes shorter than that of any of the cyclic molecule, linear molecule and bulky substituents as determined by gel permeation chromatography at a flow rate ≦1 ml/min. The difference in elution time may depend on the number of cyclodextrin threaded onto the linear molecule. Any suitable column that is commercially available can be used in gel permeation chromatography, including Bio-Rad Bio-Sil SEC 125-5, GF-710HQ, Showa Denko Co. Ltd., Sephadex G-50, G-75, G-25, G-10, Tosoh, GMPW_{XL} or the like.

The fourth aspect of a multivalently interactive molecular assembly according to the present invention may comprise a plurality of cyclodextrin, a linear molecule which is threaded through the plurality of cyclodextrins to hold them together, and capping bulky substituents at the both ends of the linear molecule, and be characterized by that, in at least two of the cyclodextrin molecules, C6 primary hydroxyl group, C2 secondary hydroxyl group and C3 secondary hydroxyl group each have a peak area which is reduced by 10 to 95% compared to that of the corresponding hydroxyl group in a cyclodextrin without substituent as determined by two-dimensional ¹H-NMR spectroscopy. This is because multiple functional groups and ligands that can interact with receptors have been incorporated into the hydroxyl group of cyclodextrin, thereby reducing the peak areas of C6 primary hydroxyl group, C2 secondary hydroxyl group and C3 secondary hydroxyl group of cyclodextrin by 10 to 95%.

In the multivalently interactive molecular assembly according to the present invention, the functional groups may preferably contain carboxyl group in terms of calcium chelating ability and trypsin inhibition activity. Calcium chelating ability and trypsin inhibition activity have been verified by the calcium binding assay and trypsin inhibition activity described below. Examples of carboxyl group-containing functional groups include carboxyalkoxy carbonyl group and preferably carboxy ethoxy carbonyl group.

Functional polyrotaxane, one example of multivalently interactive molecular assemblies according to the present invention, can be produced by synthesizing a polyrotaxane scaffold, and then introducing functional groups and/or ligands by which receptors may be caught in the hydroxyl group of α-CDs in the scaffold. Polyrotaxane, in which α-CDs are threaded through the polyoxyethylene chain capped with benzyloxycarbonyl-phenylalanine (Z-L-Phe) groups, can be prepared according to any known method. In summary, α-CD/polyoxyethylene (PEO-BA) inclusion complex was prepared by simply mixing a saturated aqueous solution of α-CD and an aqueous solution of PEO-BA. Next, succinimide ester of Z-L-Phe prepared by condensation reaction of Z-L-Phe with N-hydroxy succinimide may be allowed to react with the terminal-amino group of the inclusion complex dissolved in DMSO to synthesize a polyrotaxane scaffold containing approximately 22 α-CDs.

Introduction of ligand will be described referring to the synthesis of biotin-polyrotaxane conjugate as an example. Structural Example 2 below shows one exemplary synthesis of biotin-polyrotaxane conjugate.

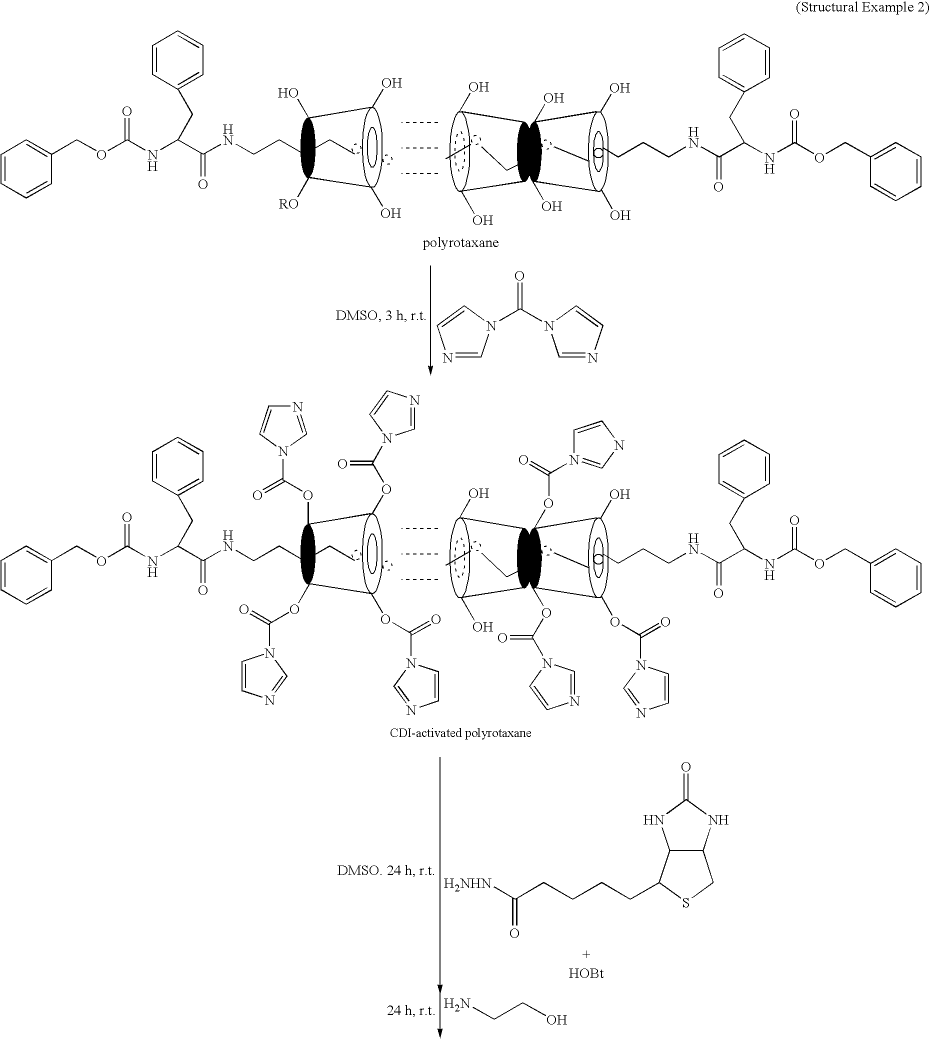

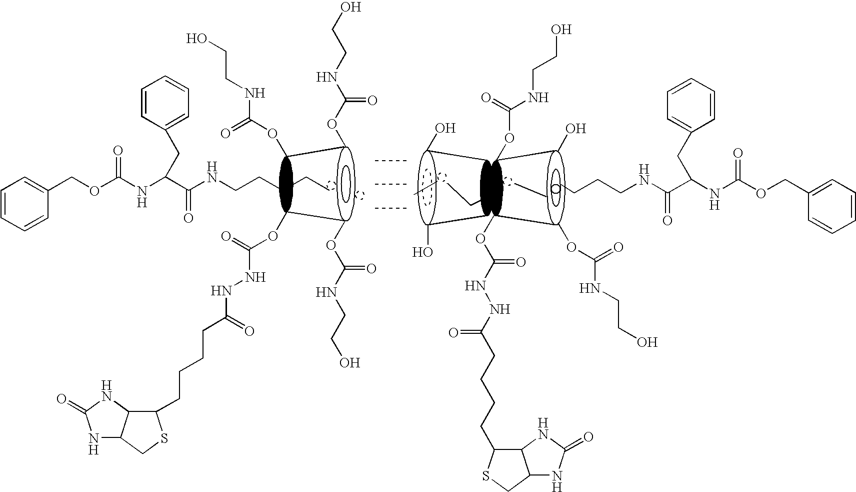

In order to introduce biotin molecules into the polyrotaxane scaffold, the hydroxyl group of α-CDs in the polyrotaxane may be activated by N,N′-carbonyldiimidazole (CDI) so that it can be reacted with the hydrazide group of biotin hydrazide.

The CDI-activated polyrotaxane (one polyrotaxane molecule contains 22 α-CDs and 0.24 mM N-acylimidazole groups) may be dissolved in 2 mL of dry DMSO, and 0.24 mM biotin hydrazide and 0.24 mM HOBt may be added to the solution under nitrogen atmosphere. The mixture solution is then stirred at room temperature for 24 hours, added dropwise with 9.9 mM 2-aminoethanol, and then stirred under the same conditions for additional 24 hours. The resulting reaction solution may be dialyzed against water through a dialysis membrane (Spectra/Pro® MWCO; 1000) and lyophilized to give biotin-polyrotaxane conjugate.

Alternatively, carboxyethyl ester-polyrotaxane complex was prepared by introducing carboxyethyl ester into polyrotaxane utilizing reaction between the hydroxyl group of the polyrotaxane and succinic anhydride in pyridine.

The multivalently interactive molecular assembly according to the present invention may have a high binding stability. Particularly, the binding stability is controllable in terms of space and time. The present inventors used SPR technique to analyze the binding/dissociation constant between the biotin-polyrotaxane conjugate and streptavidin as the model of multivalent ligands targeting to biological receptors. As the number of biotin linked to one polyrotaxane molecule increased, dissociation constant (k_{diss}) decreased rather than binding constant (k_{bind}) increased, assuming a pseudo-first-order kinetics. Dissociation did not follow the pseudo-first-order kinetics, and re-binding of biotin-polyrotaxane conjugate to the streptavidin-deposited surface was observed. The results of competitive inhibition assay showed that the biotin-polyrotaxane conjugate had a stronger inhibition activity than that of biotin-α-CD conjugate. While a biotin-α-CD conjugate may interact monovalently, a biotin-polyrotaxane conjugate containing biotin-α-CDs can interact multivalently, thereby providing multivalent kinetics. Desirable binding stability of the multivalently interactive molecular assembly can be obtained by regulating multivalency thereof when it is synthesized. Optionally, the capping bulky substituents may be designed so that they are decomposed under certain conditions to control dissociation of the cyclic molecules from the linear molecule, thereby obtaining desirable binding stability. In this way, the multivalently interactive molecular assembly according to the present invention may have a high binding stability. Particularly, the binding stability of the inventive assembly is controllable in terms of time and space.

A multivalently interactive molecular assembly according to the present invention can be used as a capturing agent that can capture its target or targets. The inventive multivalently interactive molecular assembly can be used as a capturing agent. By introducing the ones which may capture a target of capturing, either as functional group or ligand, it may be used as a capturing agent having high binding ability in which the binding ability is controllable.

A multivalently interactive molecular assembly according to the present invention can also be used as a drug carrier. The properties of the multivalently interactive molecular assembly are also useful for a drug carrier. Particularly, a drug can be introduced into the multivalently interactive molecular assembly via the functional group or ligand thereof to prepare a formulation that can then be administered to an organism. Optionally, the formulation can be designed so that the capping bulky substituents may be decomposed under certain conditions, thereby controlling the release of the drug from the polyrotaxane scaffold. Alternatively, the drug itself may act as the ligand.

A multivalently interactive molecular assembly having carboxyl group according to the present invention can be used as a calcium chelating agent or a drug enhancer. Such a multivalently interactive molecular assembly has abilities to inhibit trypsin activity and/or open the tight junction of small intestine via its calcium chelating activity and thus can be used as calcium chelating agent or drug enhancer. The multivalently interactive molecular assembly may also be useful for other biological effects of calcium chelating.

Any capturing agent can be used in the present invention which comprises at least a multivalently interactive molecular assembly according to the present invention and has an ability to capture its target or targets. An element to be introduced in the multivalently interactive molecular assembly can be suitably selected from any known materials.

Any drug carriers can be used in the present invention which comprises at least a multivalently interactive molecular assembly according to the present invention and can be bound to a drug. An element to be introduced in the multivalently interactive molecular assembly can be suitably selected from any known materials.

Any calcium chelating agents can be used in the present invention which contains at least a multivalently interactive molecular assembly according to the present invention and can chelate calcium. An element to be introduced in the multivalently interactive molecular assembly can be suitably selected from any known materials.

Any drug enhancers can be used in the present invention which comprises at least a multivalently interactive molecular assembly according to the present invention and can be used for assisting in the efficacy of drug. An element to be introduced in the multivalently interactive molecular assembly can be suitably selected from any known materials.

### EXAMPLES

[Materials]

The α-cyclodextrin (α-CD) was purchased from Bio-Research Corporation of Yokohama (Yokohama, Japan). The α-(3-aminopropyl)-ω-(3-aminopropyl) polyoxyethylene (PEO-BA: Mn=4000) was kindly supplied by Sanyo Chemical Co. (Kyoto, Japan).

The benzyloxycarbonyl-phenylalanine (Z-L-Phe), 2-ethanol, N,N′-carbonyldiimidazole (CDI), formic acid and d-biotin were purchased from Wako Pure Chemical Co. Ltd. The N-hydroxysuccinimide and 1-hydroxybenzotriazole (HOBt) were purchased from Peptide Institute, Inc. (Osaka, Japan). Streptomyces avidinii derived streptavidin was purchased from Nacalai Tesque, Inc. (Kyoto, Japan). Phosphate buffered saline (pH 7.4) containing 0.05 v/v % Tween 20 (PBS/T) (10 mM sodium phosphate, 2.7 mM calcium chloride, 138 mM sodium chloride and 0.05% Tween 20) was prepared by dissolving PBS/T powder purchased from Sigma Chemical Co. (St. Louis, USA) and kept at 4° C. until use. EZ-Link-Biotin Hydrazide™ and ImmunoPure® streptavidin were purchased from PIERCE (Rockford, USA). Biotin cuvettes for the interaction analysis system (IAsys) were purchased from Affinity Sensors Cambridge, Inc. (UK). Dimethylsulfoxide (DMSO) was purchased from Wako Pure Chemical Co., Ltd., and distilled by conventional method. The DMSO for the high performance liquid chromatography (HPLC) was purchased from Kishida Chemical Co. (Osaka, Japan). All the other chemicals used were of reagent grade.

### Example 1

Synthesis of Biotin-Polyrotaxane and Biotin-α-CD Conjugates

Polyrotaxane, in which a plurality of α-CD, is threaded through a PEO chains capped with Z-L-Phe groups by any known method. Briefly, α-CD/PEO-BA inclusion complex was prepared by simply mixing a saturated aqueous solution of α-CD and an aqueous solution of PEO-BA. Next, a succinimide ester of Z-L-Phe, which was obtained by condensation reaction of Z-L-Phe and N-hydroxysuccinimide, is reactive with the terminal amino group of the inclusion complex dissolved in DMSO. The chemical structure was determined by 750 MHz ¹H-NMR using a FT-NMR spectrometer (Varian FT-NMR Gemini 750, Palo Alto, USA). The number of α-CDs was determined to be approximately 22 based on the ¹H-NMR spectrum by comparing the integration of the signal at 4.75 (C₁H of α-CD) with one at 3.49 (CH₂CH₂O of PEO).

Next, to introduce biotin molecules into the polyrotaxane scaffold, the hydroxyl group of α-CDs in the polyrotaxane was activated by CDI so that the hydroxyl group could react with the hydrazide group of biotin hydrazide. Particularly, the polyrotaxane (13.6 μM, hydroxyl group 6.1 mM) was dissolved in 20 mL of dry DMSO, and 30.7 mM CDI was added to the solution, and then, the mixture was stirred at room temperature for 3 hours under nitrogen atmosphere. The reaction mixture was slowly added to an excess amount of ether, and the mixture was then precipitated, filtrated and dried under vacuum to give a CDI-activated polyrotaxane. The activation of the hydroxyl groups in the polyrotaxane was confirmed by calorimetric determination of imidazole after alkaline hydrolysis of N-acyl imidazole groups. The number of α-CDs per polyrotaxane molecule was approximately 22 and the degree of activation was approximately 10 per α-CD molecule. Therefore, the total degree of activation per polyrotaxane molecule is approximately 220, which indicates that hundreds of biotin can theoretically be incorporated into one polyrotaxane scaffold.

The CDI-activated polyrotaxane (one polyrotaxane contains 22 α-CDs and 0.24 mM N-acylimidazole group) was dissolved in 2 mL of dry DMSO, and 0.24 mM biotin hydrazide and 0.24 mM HOBt were added to the solution in the presence of nitrogen gas. The mixture solution was stirred at room temperature for 24 hours, added dropwise with 9.9 mM 2-aminoethanol, and stirred for 24 hours under the same conditions. The resulting reaction solution was dialyzed against water through a dialysis membrane (Spectra/Pro® MWCO; 1000) and lyophilized to give biotin-polyrotaxane conjugate.

After the reaction with biotin hydrazide, the resulting product was found to be water-soluble. It is known that hydrogen bond between the hydroxyl groups of α-CDs in polyrotaxanes exhibits limited water solubility. The reduction of the hydrogen bond by chemical modifications such as hydroxypropylation can significantly improve the water solubility of the polyrotaxane. The reduced hydrophilicity after introduction of biotin (hydrophilic ligand) appeared to be attributed to the association with alkyl chains in biotin. This was one of the reasons to carry out the chemical modification of α-CDs with 2-aminoethanol (hydroxyethylcarbamoylation). As expected, the hydrophilicity of the polyrotaxane increased after the reaction.

The polyrotaxane conjugated with biotin hydrazide and 2-aminoethanol was analyzed by gel permeation chromatography and ¹H-NMR spectroscopy. Gel permeation chromatography was performed using TSK gel G3000H_{HR}+G5000H_{HR} columns (available from Tosoh, Co., Tokyo, Japan) and elution in DMSO at flow rate of 0.8 mL/min, and detection was performed by determining angle of rotation in OR-990 (Japan Spectroscopic Co., Tokyo, Japan).

Yield: 34 mg. ¹H-NMR (DMSO-d6, ppm): δ 9.39 (d, J=2.3 Hz, 2H×11, immobilized likage —OCOHN—NHCO—), 7.38-7.16 (brm, 10H×2, aromatic ring of Z-L-Phe), 7.15-6.80 (brm, 1H×104, immobilized linkage —OCONH— of hydroxyethyl carbamoyl group), 6.40, 6.34 (s, 2H×11, NH of biotin), 4.89 (brm, 6H×20, C₁H, C₆H₂, C₄H and C₂H of α-CD), 3.51 (s, 4H×90, CH₂CH₂O of PEO), 3.04 (brm, 4H×104, CH₂ of hydroxyethyl carbamoyl group), 2.82 (dd, J=4.5, 7.5 Hz 1H×11, C_{ε}H of biotin), 2.58 (d, J=12.8 Hz, 1H×11, C_{ε}H′ of biotin), 2.08 (m, 2H×11, C_{α}H of biotin), 1.63-1.23 (m, 6H×11, C_{β}H/C_{γ}H/C_{δ}H of biotin). The number of α-CDs and immobilized biotin were determined from the 750 MHz ¹H-NMR spectrum.

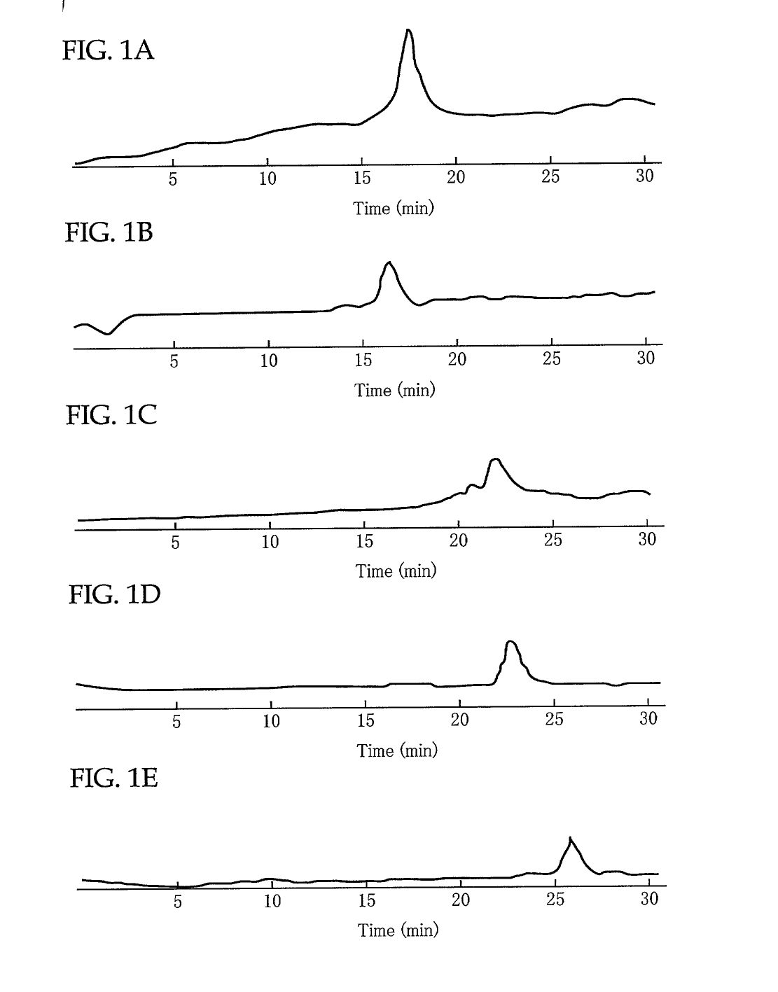

FIGS. 1A to 1E show the results of gel permeation chromatography: FIG. 1A for purified biotin-polyrotaxane conjugate; FIG. 1B for hydroxyethyl carbamoyl-polyrotaxane; FIG. 1C for biotin-α-CD conjugate (0.6 biotin per α-CD); FIG. 1D for α-CD; and FIG. 1E for d-biotin. The peak attributed to the biotin-polyrotaxane conjugate was detected as a single peak within its elution time, which was significantly shorter than that of any of the biotin-α-CD conjugate, α-CD and d-biotin. Further, the elution time profile of the biotin-polyrotaxane conjugate was very close to that of hydroxyethylcarbamoyl-polyrotaxane. These results indicate that the product obtained was a polyrotaxane derivative with no contamination.

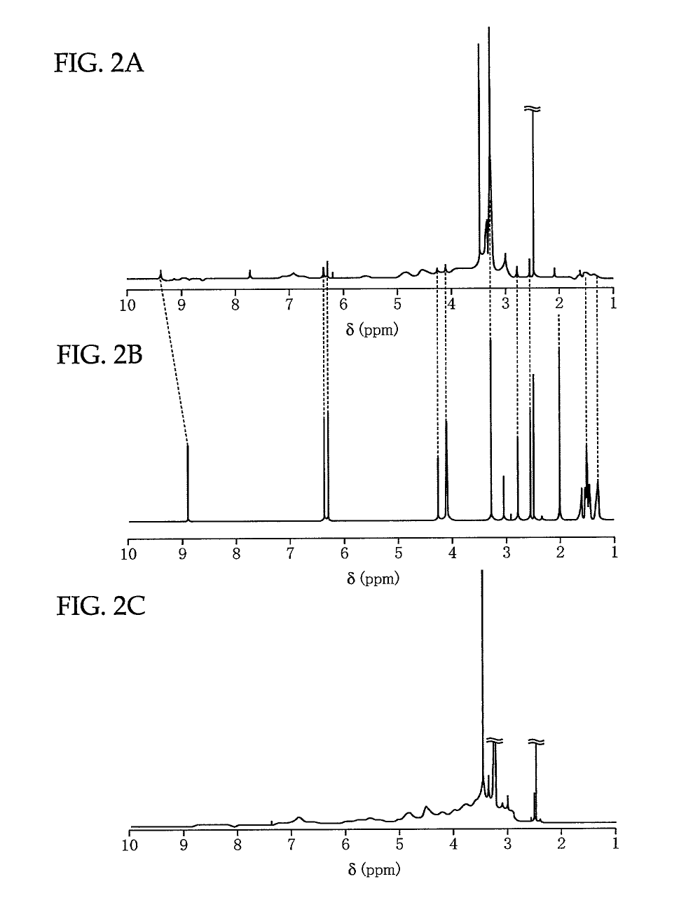

In order to confirm the chemical composition of the polyrotaxane derivative (i.e., biotin-polyrotaxane conjugate), its ¹H-NMR spectrum was compared with those of biotin hydrazide and hydroxyethylcarbamoyl-polyrotaxane (FIGS. 2A to 2C). FIGS. 2A, 2B and 2C show results for biotin-polyrotaxane conjugate, d-biotin, and hydroxyethylcarbamoyl-polyrotaxane, respectively. The peaks attributed to d-biotin and hydroxyethylcarbamoyl-polyrotaxane were confirmed in the analysis of biotin-polyrotaxane conjugate. The peak attributed to the hydrazide groups (δ=8.91 in FIG. 2B) exhibited a downfield shift (δ=9.39 in FIG. 2A). This peak shift shows that the d-biotin hydrazide was introduced to the hydroxyl groups of α-CDs in the polyrotaxane via carbamoyl linkages. These results indicate that the biotin was conjugated with polyrotaxane and the supramolecular structure of the latter was maintained after the biotin immobilization.

One polyrotaxane molecule contained 20 α-CDs, 11 biotins and 104 hydroxyethylcarbamoyl groups as determined based on the ¹H-NMR spectra, indicating that about one biotin molecule was present for two α-CD molecules.

The conformation of the synthesized biotin-polyrotaxane conjugate in an aqueous solution was analyzed by two-dimensional nuclear Overhauser effect spectroscopy (2D NOESY). There were no correlated peaks between the peaks of d-biotin (NH, C_{α-δ}H, C_{ε}H, C_{ε}H′, C_{ζ}H, C_{ζ}H′) and those of hydroxyethylcarbamoyl-polyrotaxane (aromatic ring of Z-L-Phe, O₆H, C₅H, C₆H₂, C₄H, C₃H, C₂H and C₁H of α-CD, CH₂CH₂O of PEO, and CH₂ of hydroxyethylcarbamoyl group), although several correlated peaks were observed between the glucose units of α CDs, presumably due to configurational changes after conjugation. These results indicate that the biotin molecules in the conjugate were exposed to a water-soluble environment without associating with each other.

### Example 2

Analysis of Biotin-Polyrotaxane Conjugate Binding to Streptavidin-Immobilized Surface Using Surface Plasmon Resonance Analyzer (SPR Analyzer)

SPR experiments were carried out using an IAsys device (IAsys Auto+, Affinity Sensors Cambridge Inc. UK) that can quantify a wide range of biomolecular interactions by a resonance mirror biosensor. The IAsys device temperature was set at 25° C. The resonant layer of biotin cuvette was washed with 40 μL of PBS/T and allow to settle for 10 min for equilibration. During this equilibration, streptavidin was dissolved in PBS/T to 1 mg/ml. The solution of streptavidin in PBS/T (20 μL) was added to the PBS/T in the cuvette and left to stand for 10 min to allow the streptavidin to deposit to the biotin-immobilized surface. After washing the cuvette with 50 μL of PBS/T three times, the cuvette was left to stand for 3 min to stabilize the base line. The density of the deposited streptavidin was determined from the sensorgram obtained based on the IAsys calibration curve. After equilibrating the streptavidin-deposited surface with 45 μL of PBS/T, the biotin conjugate dissolved in PBS/T (50 nM biotin in the conjugate) was added to PBS/T in the cuvette, and the binding was then monitored for 10 min. Next, the cuvette was washed with 50 μL of PBS/T, and monitored for additional 5 minutes to observe dissociation. Finally, 1M formic acid was added to the surface to disrupt the biotin-streptavidin binding, then the cuvette was washed with PBS/T three times. The same procedure was repeated but using various concentrations of the biotin-polyrotaxane conjugate. The resulting sensorgram was analyzed based on pseudo-first-order kinetics to obtain kinetic parameters.

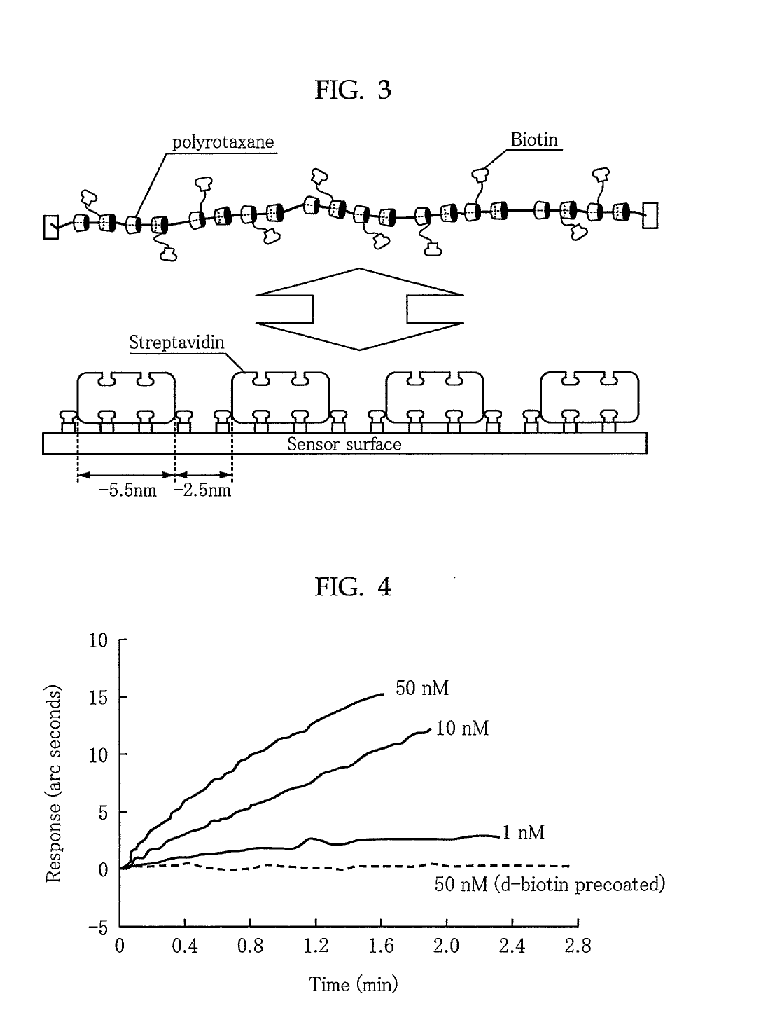

As described above, streptavidin tetramer was deposited on the biotin-immobilized IAsys cuvette. The density of the deposited streptavidin was 2.5×10⁻⁵ nmol/mm², which means that streptavidin tetramer was deposited on the surface at a density of 1 streptavidin molecule/64.2 nm², and that the average distance between two adjacent streptavidin tetramer molecules on the surface was therefore approximately 8.0 nm. Based on the estimated size of streptavidin tetramer (5.5 nm), a schematic view of the streptavidin-deposited surface is shown in FIG. 3.

Since the depth of α-CD is 0.7 nm and the stoichiometric number of α-CDs in a PEO chain (Mn: 4,000) is 45, the theoretical length of the polyrotaxane rod can be estimated to be 32 nm. Considering the number of α-CD in the conjugate (approximately 20) and the density of the streptavidin deposited on the surface, the potential for interaction was between four streptavidin tetramers and one conjugate molecule (FIG. 3). Binding curves showing binding of the conjugate to the streptavidin-deposited surface are shown in FIG. 4. The concentration was calculated on a biotin basis. The response increased as the concentration of the conjugate injected to the streptavidin-deposited surface increased from 1 nM to 50 nM. However, such an increase in the response was not observed when a streptavidin surface overcoated with 1 mM biotin was used. These results indicate that the biotin in the conjugate was actually recognized by streptavidin.

### Example 3

Effect of the Number of Biotin Molecule in the Conjugate on Binding/Dissociation Constant

The above-described experiments showed that biotin-polyrotaxane conjugate containing approximately 11 biotin molecules was recognized by streptavidin-deposited surface. It should be noted that streptavidin does not bind to polyrotaxane itself. Next, how the number of biotin contained in one conjugate molecule affects the binding/dissociation constant associated with the multivalency of the biotin-polyrotaxane conjugates was examined.

The number of biotin contained in one polyrotaxane molecule could be varied by changing the molar ratio between CDI-activated polyrotaxane and EZ-Link biotin hydrazide (Table 2).

TABLE 2
Synthesis of biotin conjugate for kinetics analysis

| | | Number | Number | Number | |
|---|---|---|---|---|---|
| | Molar | of | of α | of | |
| Sample | Ratio *2 | biotin/m | -CD/mol | HEC/mol | |
| Code *1 | [Bio]/[Im] | ol | *3 | *3 | Total Mn |
| 11BIO-α/ | 0.5 | 11 | 20 | 104 | 33,300 |
| E4-PHE- | | | | | |
| Z | | | | | |
| 35BIO-α/ | 1 | 35 | 22 | 113 | 43,500 |
| E4-PHE- | | | | | |
| Z | | | | | |
| 78BIO-α/ | 2 | 78 | 22 | 188 | 60,900 |
| E4-PHE- | | | | | |
| Z | | | | | |
| 1BIO-α | 1 *4 | 1 | — | 4 | 1,480 |

In Table 2, BIO-α/E4-PHE-Z and BIO-α CD represent biotin-polyrotaxane and biotin-α-CD conjugates, respectively (*1). In the first column in Table 2, information regarding the number of CDs per conjugate and the functional group(s) or ligand(s) linked thereto are provided before a (/) mark. For example, 11BIO-α/E4-PHE-Z means that the sample conjugate contains 11 biotins as functional groups and α-CDs as the cyclic molecule. Information regarding the linear molecule which is threaded through the cyclic molecules and the capping bulky substituents are provided after the (/) mark. For example, 11BIO-α/E4-PHE-Z means that the sample conjugate contains a polyethylene glycol (PEG) having an average molecular weight of 4,000 capped with benzyloxycarbonyl-L-phenylalanine (Z-PHE) groups at its both ends. [Bio] and [Im] refer to the concentrations of EZ-Link™ biotin hydrazide and N-acyl imidazole group (the activated hydroxyl group of α-CDs in the polyrotaxane), respectively (*2). The number of α-CD and of hydroxyethylcarbamoyl group (HEC) were calculated based on the 750 MHz ¹H-NMR spectrum (*3). One N-acylimidazole group per α-CD has been introduced (*4).

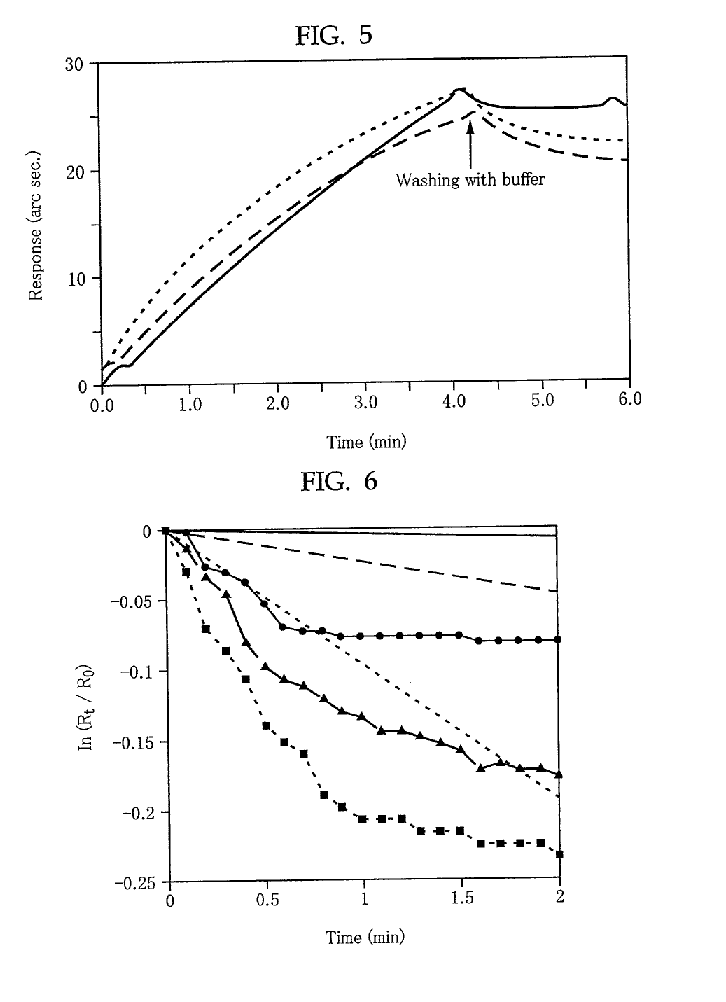

The SPR sensorgram showing the binding/dissociation of 11BIO-α/E4-PHE-Z, 35BIO-α/E4-PHE-Z and 78BIO-α/E4-PHE-Z to and from the streptavidin-deposited surface is shown in FIG. 5. The concentration of biotin in the conjugate is 50 nM and the fine and rough dotted lines and the solid line represent reactions of 11BIO-α/E4-PHE-Z, 35BIO-α/E4-PHE-Z and 78BIO-α/E4-PHE-Z, respectively, in FIG. 5. Injection of each conjugate onto the streptavidin-deposited surface increased reaction though no qualitative difference was detected in the binding reaction depending on the difference in the number of biotin in the conjugate. In order to dissociate biotin-polyrotaxane conjugate, the solution was replaced by PBS/T buffer, and dissociation curves were obtained 4 minutes after injection (FIG. 5). It seemed that conjugate with larger number of biotin had a gentler slope in its the dissociation curve. These results suggest that the number of biotin affected the dissociation rather than the binding.

In order to dissect the binding/dissociation constant, the binding curves in FIG. 5 were analyzed in terms of the pseudo-first-order kinetics, which was based on the interaction between ligand (L: in this case biotin-polyrotaxane conjugate) and immobilized receptor (R: in this case streptavidin):

$$\begin{matrix}L + R {⇄}_{{k}_{diss}}^{{k}_{bind}} LR , & {K}_{α} = {k}_{bind} / {k}_{diss}\end{matrix} \qquad ( 3 )$$

wherein k_{bind} is a binding constant, k_{diss} a dissociation constant, and K_{α} an association equilibrium constant. Rₜ (which represents an SPR response at time t) and dR/dt (the binding rate) can be used in the following kinetics of interaction:

dR/dt=k_{bind}C_{L}(Rₘₐₓ−Rₜ)−k_{diss}Rₜ (4a)

Rₜ=R_{eq}[1−exp(−k_{obs}t)] (4b)

k_{obs}=k_{bind}C_{L}+k_{diss} (4c)

wherein C_{L} is the concentration of conjugate injected (in this case, the biotin bound), Rₘₐₓ the maximum binding response and k_{obs} the pseudo-linear rate of the binding. The k_{obs} value was calculated for the conjugates from their binding curves obtained by changing the conjugate concentration. Plot of k_{obs} as a function of biotin concentration in the conjugate [Eq. (4c)] was well fitted to a linear line (r²=0.987 to 0.998) using the linear least-square method. Thus, k_{bind} and k_{diss} were calculated using Equation (4c). Table 3 summarizes the kinetic parameters for 11BIO-α/E4-PHE-Z, 35BIO-α/E4-PHE-Z and 78BIO-α/E4-PHE-Z.

TABLE 3

| | k_{bind} | k_{diss} | |
|---|---|---|---|
| Binding | (×10⁴M⁻¹sec⁻¹) | (×10⁻³sec⁻¹) | kₐ (×10⁷M) |
| 11BIO-α/E4-PHE-Z | 13.8 | 1.6 | 8.6 |
| 35BIO-α/E4-PHE-Z | 1.7 | 0.39 | 4.4 |
| 78BIO-α/E4-PHE-Z | 5.2 | 0.052 | 100.0 |

These data show that the k_{diss} value dramatically decreased as the number of biotin in the conjugate increased while the value in the k_{bind} remained about the same. Accordingly, the Kₐ value of 78BIO-α/E4-PHE-Z was about 12-fold higher than that of 11BIO-α/E4-PHE-Z and about 22-fold higher than that of 35BIO-α/E4-PHE-Z. It is known that high-affinity mediated by multivalent interaction is due to decrease in the dissociation rate of multivalent ligand, rather than to increase in the binding rate. Therefore, decreased in the k_{diss} value indicates that biotin (ligand) in the conjugates bind multivalently to the deposited streptavidin.

However, Winzor et al. suggested that the pseudo-first-order kinetics is not suitable for the analysis of multivalent interaction. Therefore, whether the dissociation constant follows the pseudo-first-order kinetics or not was examined.

To evaluate the dissociation constant, the classical expression was considered for the dissociation based on the pseudo-first-order kinetics. CL in Equations (4a) to (4c) should be zero for the dissociation process since the buffer containing the conjugates in the SPR cuvette was replaced by the buffer without conjugate. Thus, the dissociation constant can be expressed by the following equations:

Rₜ·R₀=exp(−k_{diss}t) (5a)

ln(Rₜ/R₀)=−k_{diss}t (5b)

where R₀ is the degree of SPR response at the start point of the buffer injection. There should be a linear relationship between ln (Rₜ/R₀) and time if the dissociation constant follows Equation (5b). FIG. 6 shows time dependence of ln (Rₜ/R₀) for 11BIO-α/E4-PHE-Z (▪ in FIG. 6), 35BIO-α/E4-PHE-Z (▴ in FIG. 6) and 78BIO-α/E4-PHE-Z (● in FIG. 6). In FIG. 6, the fine dotted line, the rough dotted line and the solid line indicate theoretical linear lines for 11BIO-α/E4-PHE-Z, 35BIO-α/E4-PHE-Z and 78BIO-α/E4-PHE-Z, respectively, obtained using the corresponding k_{diss} values in Table 3. The experimental plots in FIG. 5, determined based on the text data of the SPR sensorgram, did not conform to the linear lines predicted by the logarithmic function of Equation (5b). All the conjugates (including those with larger number of biotin) had gentle slopes in the carves after 0.6-0.8 min. These results suggest that the biotin-polyrotaxane conjugates may re-bind with the streptavidin-deposited surface, which strongly supports their multivalent property. The linear lines without any marks▪, ▴ or ● in FIG. 6 represent the theoretical relationship obtained by applying the k_{diss} values in Table 3 to Equation (5b). These lines did not conform to the experimental plots. However, the slopes of the linear lines seem to substantially conform to those of the experimental plots after the re-binding, which shows that the calculated k_{diss} values in Table 3 may represent the multivalent kinetics.

### Example 4

Competitive Inhibition of Streptavidin-Biotin Binding by the Multivalent Inhibitor (Biotin-Polyrotaxane) and the Monovalent Inhibitor (biotin-α-CD)

In order to compare the kinetics of the biotin-polyrotaxane conjugates with that of the biotin-α-CD conjugate, firstly, binding of biotin-α-CD conjugate (1BIO-α CD in Table 2) to the streptavidin-deposited surface was analyzed by SPR. Unfortunately, significant SPR sensorgram could not be obtained for 1BIO-α CD due to its low molecular weight. According to the current SPR technology, it is difficult to detect the interaction between a low-molecular-weight ligand (Mn<˜5000) and its immobilized receptor since the size of the molecule formed on the sensor surface by complexing of such a small ligand with the receptor is too small to change the refractive index. As an alternative, we carried out a competitive inhibition assay for quantifying the substance that inhibits the interactions between a soluble receptor and its immobilized ligand (see, Mammen, et al., Angew. Chem. Int. Ed. 37 (1998) 2754-2794; Mann et al., J. Am. Chem. Soc. 120 (1998) 10575-10582; Sigal et al., J. Am. Chem. Soc. 118 (1996) 3789-3800).

Competitive Assay

Competitive assay was performed using biotin-polyrotaxane and biotin-α-CD conjugate according to the method reported by Kiessling et al. The biotin-polyrotaxane or the biotin-α-CD conjugate (which corresponds to 78BIO-α/E4-PHE-Z or 1BIO-α CD in Table 2, respectively) was dissolved in PBS/T to a biotin concentration of 1 mM, and the other 5 dilution samples of 0.25, 0.5, 1.0, 10 and 100 μM were additionally prepared.

One hundred micro liters of solution of streptavidin in PBS/T (0.1 mg/ml, 1.5 μM) was added to each sample solution (0.9 ml) and mixed well using a mixer. These solutions were incubated for 1 hour at room temperature. Each of the resulting solutions (5 μL) were injected to the resonant layer of a biotin cuvette that was equilibrated with 45 μL of PBS/T (10 times dilution of the sample solution). The SPR reaction was monitored in the same manner as in the binding analysis using the biotin cuvette. To obtain the inhibition constant (Kᵢ), the SPR data were analyzed by solution competition equation using a modified rectangular hyperbolic relationship:

f=[I]/([I]+Kᵢ(1+F/K_{d})) (6)

where f is fractional inhibition that is calculated using equilibrium values obtained in the absence of inhibitor (biotin conjugate), [I] the concentration of inhibitor (biotin residue), F the concentration of free binding sites available for the streptavidin, and K_{d} the dissociation constant of streptavidin from the surface. To determine the F and K_{d} values, data on the binding of streptavidin to the surface of the biotin cuvette (final streptavidin concentrations in the cuvette: 0.1 to 10 μg/ml) were collected, and its response values were fitted to the following rectangular hyperbolic equation:

R_{eq}=Rₘₐₓ[SV]/(Kd+[SV]), Kd=Rₘₐₓ/2 (7)

where R_{eq} is equilibrium response, Rₘₐₓ the maximum binding response of the streptavidin, and [SV] the concentration of streptavidin. The F and Kd values calculated were found to be 7.9±0.46 nM and 3.2±0.92 nM, respectively. The Kᵢ values for 78BIO-α/E4-PHE-Z and 1BIO-α CD were derived by a curve fitting the obtained plots off and [I] based on Equation (6) using Microcal Origin 6.0 software.

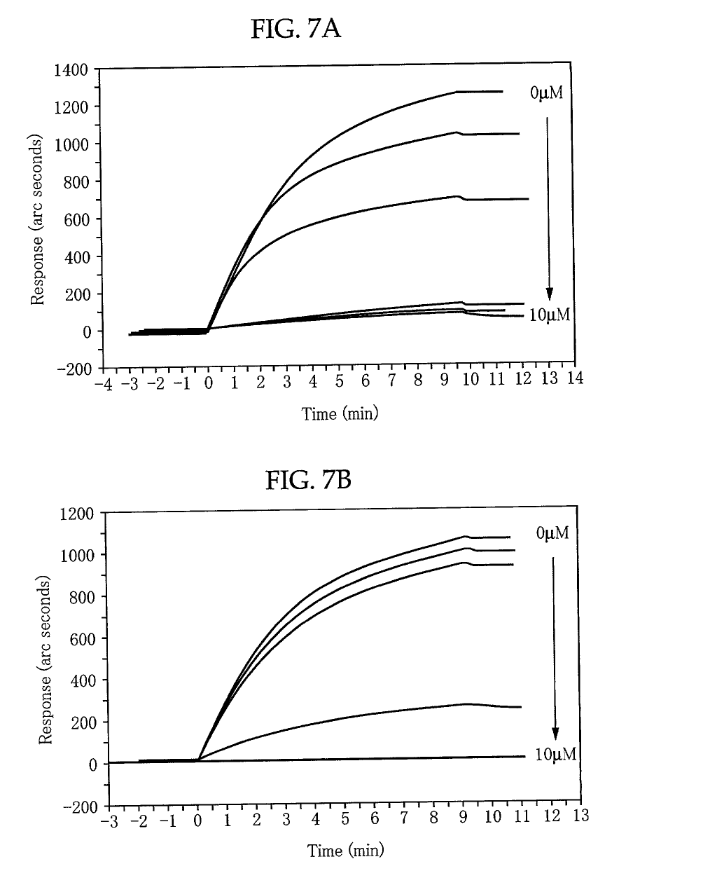

Concentration-dependent inhibition curves obtained by measuring the binding of 0.015 μM streptavidin to the surface in the presence of various concentrations of the biotin-polyrotaxane conjugates or biotin-α-CD (1BIO-α CD) conjugate are shown in FIGS. 7A and 7B. Particularly, FIGS. 7A and 7B show inhibition curves illustrating the inhibition of 0.015 μM streptavidin binding to a biotin-immobilized sensor surface by biotin in 0, 0.025, 0.05, 0.1, 1 and 10 μM conjugates. FIG. 7A shows inhibition by 78BIO-α/E4-PHE-Z while FIG. 7B shows inhibition by 1BIO-α-CD. The R_{eq} value was 1,000 to 1,200 arc/second in the absence of conjugate and decreased as the concentration of conjugates increased (from 0 to 10 μM as biotin basis) for the both conjugates. Within lower concentration range (0.025-0.1 μM), the R_{eq} value for 78BIO-α/E4-PHE-Z was relatively smaller than that for 1BIO-α CD, suggesting that the binding ability of 78BIO-α/E4-PHE-Z to streptavidin in solution was superior to that of 1BIO-α CD.

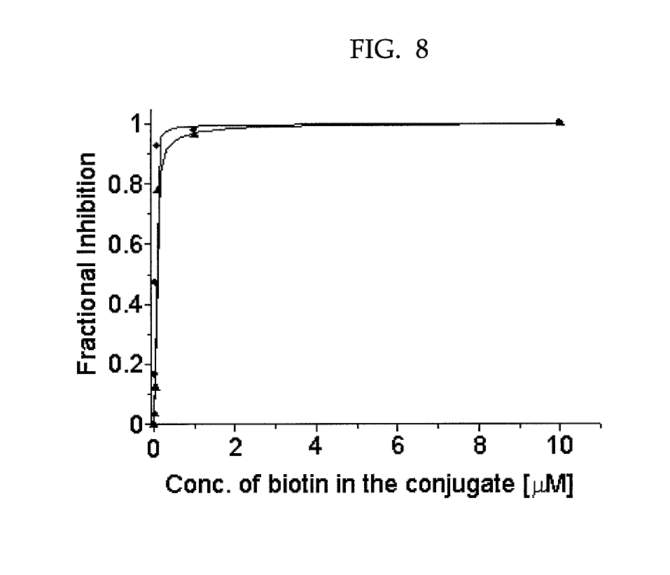

The inhibition constant Kᵢ value indicating inhibition of streptavidin binding to the biotin-immobilized surface by conjugate was calculated by using the plot of fractional inhibition vs. the conjugate concentration (FIG. 8) and Equation (6). In FIG. 8, ● and ▴ indicate the results for 78BIO-α/E4-PHE-Z and 1BIO-α CD, respectively. The Kᵢ values for 78BIO-α/E4-PHE-Z and 1BIO-α CD were 2.13±0.25 and 9.48±1.08 nM, respectively. These results suggest that the biotin-polyrotaxane conjugate had from 4- to 5-fold higher activity than that of the biotin-α-CD conjugate.

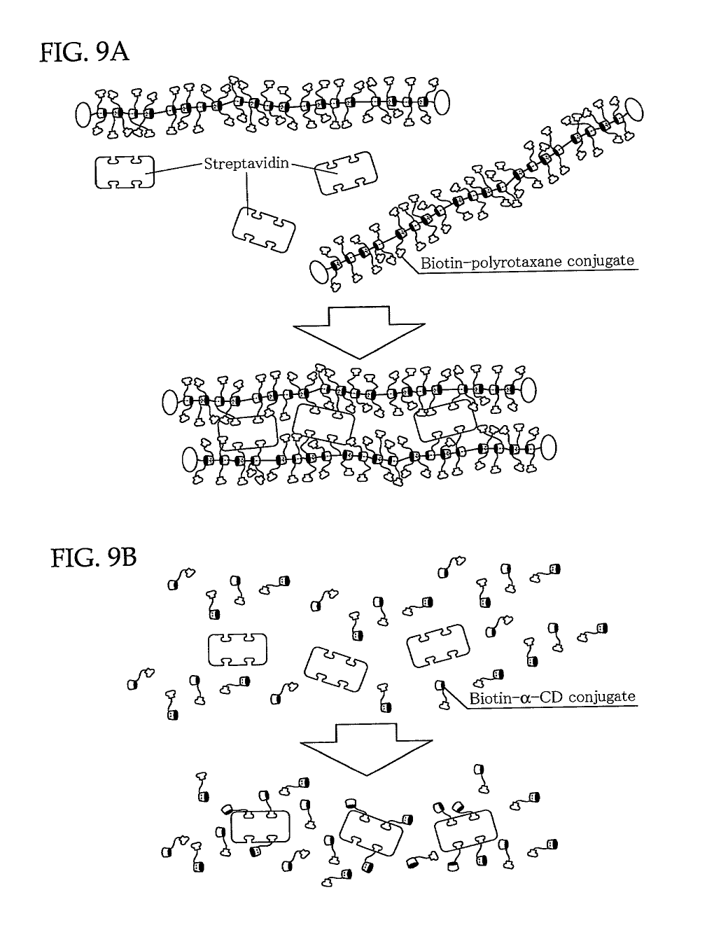

Streptavidin is known to form tetramer that has four binding sites, and its size is assumed to be 5.5 nm. It can be assumed that the depth of α-CD is 0.7 nm and the stoichiometric number of α-CDs which can be threaded onto one PEO chain (Mn: 4,000) is approximately 45. The theoretical length of polyrotaxane rod may therefore be 32 nm. Since one 78BIO-α/E4-PHE-Z molecule contains approximately 22 a CDs, it can be assumed that the majority of the biotin-polyrotaxane conjugate can span two of the binding sites of streptavidin, thereby noncovalent cross-linking streptavidin (FIG. 9A). On the other hand, 1BIO-α CD cannot span any binding sites (FIG. 9B). Therefore, it can be considered that the enhanced inhibitory activity of the biotin-polyrotaxane conjugate may be attributed to its linear structure in which multiple biotin-conjugated α-CDs are bound to the PEO chain (polyrotaxane backbone) so that the biotin-conjugated α-CDs are arranged in a line along the PEO chain.

### Example 5

Synthesis and Characterization if Carboxyethylester Polyrotaxane (a Novel Calcium Chelating Polymer)

Polyrotaxane was allowed to react with succinic anhydride in pyridine to introduce carboxyethylester group into the polyrotaxane via reaction between the hydroxyl group of the polyrotaxane and the succinic anhydride. This reaction was selected because the nucleophilic reaction using anhydride is known to maintain the structure of polyrotaxane (Watanabe et al., J. Biomater. Sci. Polym. Edn. 10 (1999) 1275-1288).

Particularly, carboxyethylester-polyrotaxane was synthesized according to a modified version of the method described in Tanaka et al., J. Antibiotics, 47 (1994) 1025-1029. Synthesis of carboxyethylester-polyrotaxane (132CEE-α/E4-PHE-Z) is shown below:

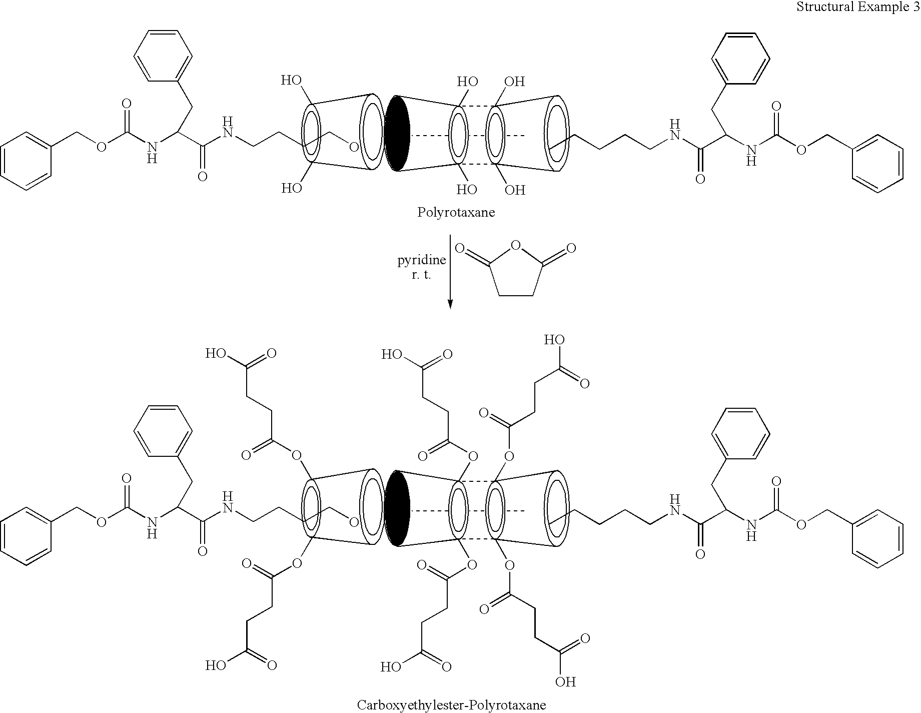

In Structural Example 3 above, polyrotaxane comprising a PEO-BA chain, multiple α-CDs threaded onto the PEO-BA chain capped with Z-L-Phe groups was synthesized in the same way as the procedure described above for the biotin-conjugated polyrotaxane. The polyrotaxane obtained (which contained 30 α-CDs as determined by ¹H-NMR assay) (6.03×10⁻⁶ mole) and succinic anhydride (3.26×10⁻⁶ mole) (available from Wako Pure Chemical Co. Ltd.) were dissolved in pyridine anhydride and stirred at room temperature. The reaction mixture was washed three times with an excess amount of ether. Precipitate was collected by centrifugation and dried under reduced pressure to give carboxyethylester-polyrotaxane (CEE-α/E4-PHE-Zs). CEE-α-CD was synthesized in the same manner as CEE-polyrotaxane.

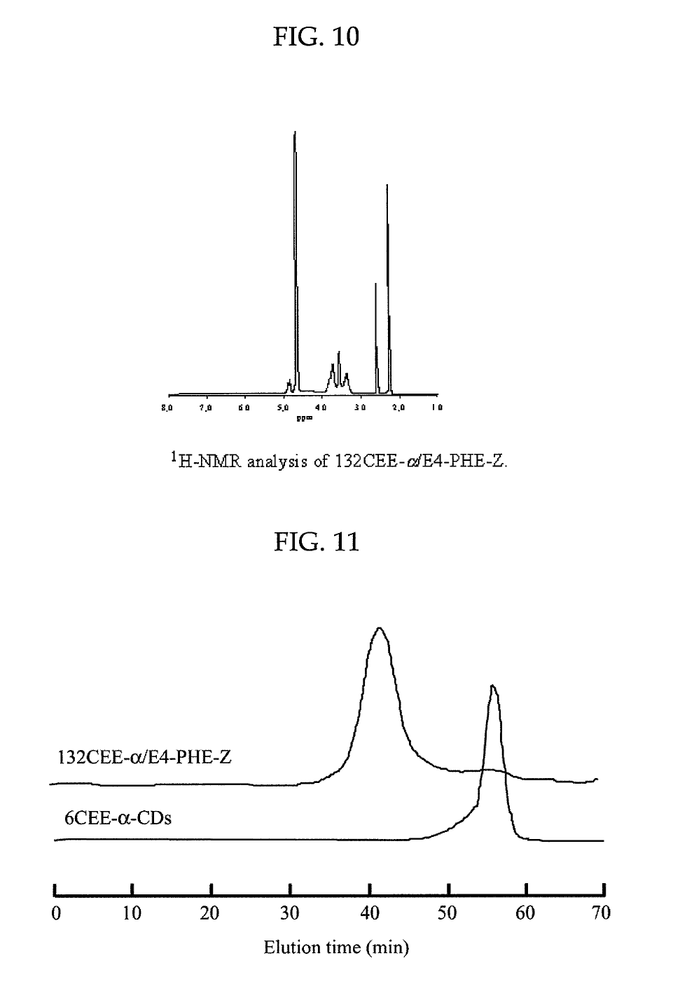

From the ¹H-NMR spectrum of the recovered sample, all the peaks were identified to be attributed to α-CDs, PEG-terminal group and carboxyethyl carbonyl group (FIG. 10). Further, a single peak was detected for 132CEE-α/E4-PHE-Z, and its elution time was much shorter than that of 6CEE-α-CD as determined by gel permeation chromatography (GPC) analysis (FIG. 11). These results indicate that the structure of polyrotaxane was maintained after the chemical modification. Table 4 shows the results of synthesis. The Mn of the PEG in CEE-α/E4-PHE-Zs is 4000 (*1). The molar ratio between succinic anhydride and α-CD is 1.0 (*2). The number of CEE group was determined based on the ¹H-NMR spectrum (*3).

TABLE 4

| | | The | The | The |
|---|---|---|---|---|
| | | number of | number of | number of |
| Sample | Reaction | α-CD/ | CEE | CEE |
| Code | time | polyrotaxane | group/ | group/PRX |
| *1 | (hour) *2 | *2 | α-CD *3 | *3 |
| 33CEE-α/ | 2 | 22 | 2 | 33 |
| E4-PHE-Z | | | | |
| 68CEE-α/ | 6 | 22 | 3 | 68 |
| E4-PHE-Z | | | | |
| 132CEE-α/ | 24 | 22 | 6 | 132 |
| E4-PHE-Z | | | | |
| 6CEE-α/ | 1 | — | 6 | — |
| CD | | | | |

As shown in Table 4, the number of CEE group can be controlled by changing the reaction time. The molar ratio between the hydroxyl group of α-CDs in the polyrotaxane and succinic anhydride had no effect on the number of CEE group. The maximum number of CEE group incorporated was 132, indicating that CEE groups were introduced to all the primary hydroxyl groups of α-CDs in the polyrotaxane. All the primary hydroxyl groups of α-CDs in 6CEE-α-CD were also modified (Table 2).

The degree of substitution with CEE group was estimated from the ratio of the peak for methylene group of the CEE group (2.28 ppm) to C(1)H (4.88 ppm) of α-CD on the ¹H-NMR spectrum.

CEE-Polyrotaxane

¹H-NMR (D₂O+NaOD, ppm): δ 7.35-7.18 (aromatic ring of Z-L-Phenylalanine), 4.88 (C(1)H of α-CD), 4.00-3.30 (C(3)H, C(5)H, C(6)H, C(4)H and C(2)H of α-CD), 3.58 (methyl group of PEO), 2.28 (methyl group of CEE), CEE-α-CD

¹H-NMR (D₂O+NaOD, ppm): δ 4.88 (C(1)H of α-CD), 4.00-3.30 (C(3)H, C(5)H, C(6)H, C(4)H and C(2)H of α-CD), 3.58 (methyl group of PEO), 2.50-2.00 (methyl group of CEE)

Next, the effects of the supramolecular structure of polyrotaxane on its solubility at various pH conditions, calcium chelating ability and trypsin inhibition were determined using 132CEE-α/E4-PHE-Z and 6CEE-α-CD.

### Example 6

Solubility in a Buffer at Various pH Conditions

The solubility of 132CEE-α/E4-PHE-Z and 6 CEE-α-CD (Table 1) in PBS was determined at various pH conditions by phenol-sulfuric acid method. An excess amount of 132CEE-α/E4-PHE-Z or 6CEE-α-CD was suspended in a 0.5M phosphate buffered saline (PBS). The pH was adjusted by adding 5M NaOH solution. Phenol-sulfuric acid method was performed according to the previous method (Watanabe et al., Chem. Lett (1998) 1031-1032). Based on the glucose (monosaccharide) content quantified by phenol-sulfuric acid method, the concentration of 132CEE-α/E4-PHE-Z and the number of α-CDs per polyrotaxane were calculated.

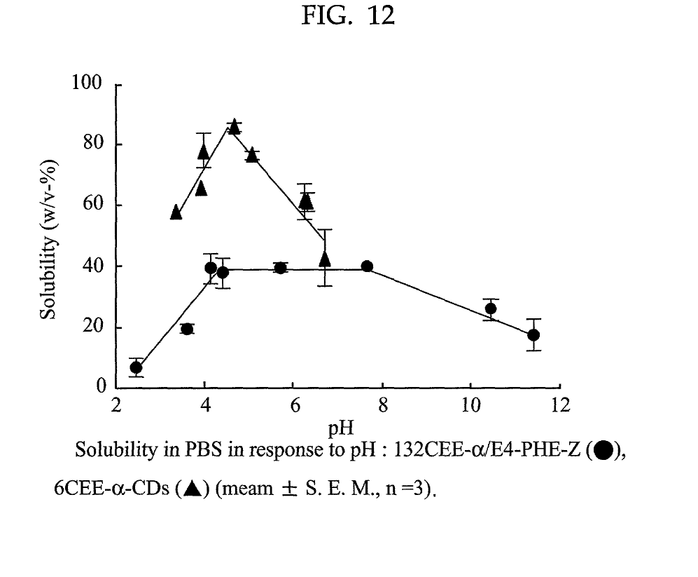

FIG. 12 shows the solubility of 132CEE-α/E4-PHE-Z (●) and 6CEE-α-CD (▴) in PBS at various pH conditions. The solubility of 132CEE-α/E4-PHE-Z and 6CEE-α-CD increased at up to pH 4 due to the ionization of carboxyl groups. The solubility of 132CEE-α/E4-PHE-Z substantially remained at a constant level between pH 4 and pH 8 and then slowly decreased until pH11. Neutralization by sodium ion would explain this decrease. Since the sodium hydroxide solution was added to the mixture in order to adjust the pH of the solution of 132CEE-α/E4-PHE-Z, the concentration of sodium ion and pH increased. It can be assumed that the neutralization of carboxyl group by the sodium ion reduced the hydration of 132CEE-α/E4-PHE-Z. The effect of such neutralization has been reported on carbopol (Unlu et al., Pharm. Acta. Helv., 67 (1992) 5-10). Unlike 132CEE-α/E4-PHE-Z case, the solubility of 6CEE-α-CD decreased from pH 5. Since a smaller peak was detected for CEE group on the NMR spectrum, it can be assumed that the solubility of 6CEE-α-CD was decreased from pH 5 because the group were included into the cavity of α-CD and formed a complex with the α-CD. The solubility of 132CEE-α/E4-PHE-Z was lower than that of 6CEE-α-CD, indicating that hydrogen bond between unmodified hydroxyl groups (secondary hydroxyl group) in 132 CEE-α/E4-PHE-Z reduced the solubility. The ester bond of these CEEs was found to be stable at pH 6-8 for 2 months or more.

### Example 7

Polyacrylic acid (PAA, Mw=25000) and calcium chloride were purchased from Wako Pure Chemical Co. Ltd. 2-N-morpholinoethanesulfonic acid (MES) was purchased from Nacalai Tesque, Inc. (Osaka Japan).

Calcium Binding Assay

Calcium binding assay was performed to examine the effect of the polyrotaxane structure on calcium ion chelating

132CEE-α/E4-PHE-Z (0-1.29×10⁻⁴ mole) or 6CEE-α-CD (0-3.23×10⁻⁴ mole) was dissolved in an aqueous solution of 50 mM 2-N-morpholinoethane sulfonic acid (MES) adjusted to pH 6.7 with 1M potassium hydroxide containing 13 mM calcium chloride (MES/KOH buffer, pH 6.7), and stirred at room temperature for 2 hours. The concentration of free Ca²⁺ ion ([Ca²⁺]_{free}) was determined using a calcium ion-sensitive electrode (HORIBA, Ltd., Japan). The concentration of chelated calcium ion ([Ca²⁺]_{bind}) was calculated using the following equation:

[Ca²⁺]_{bind}=[Ca²⁺]ₜₒₜₐₗ−[Ca²⁺]_{free}

where ([Ca²⁺]ₜₒₜₐₗ) is the total concentration of Ca²⁺.

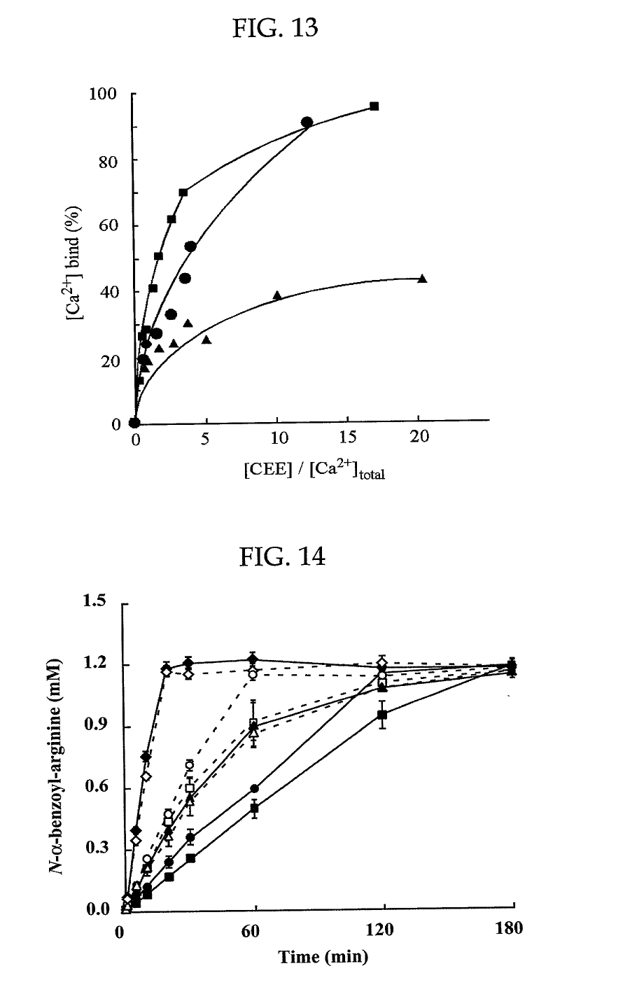

The deleted calcium ion ([Ca²⁺]_{bind}) bound to 132CEE-α/E4-PHE-Z (●), polyacrylic acid (PPA) (▪) or 6CEE-α-CD (▴) is shown as a function of the ratio of CEE concentration to total calcium ion concentration ([CEE]/[Ca²⁺]ₜₒₜₐₗ) in FIG. 13. [Ca²⁺]_{bind} of PAA increased in proportion to [CEE]/[Ca²⁺]ₜₒₜₐₗ by the value around 2-3, and then slowly increased as [CEE]/[Ca²⁺]ₜₒₜₐₗ increased. This result was consistent with the previous report by Kriwet and Kissel, Int. J. Pharm. 127 (1996) 135-145.

The 132CEE-α/E4-PHE-Z chelated calcium ion up to 90% as [CEE]/[Ca²⁺]ₜₒₜₐₗ increased, indicating that calcium ion chelating capacity of 132CEE-α/E4-PHE-Z is equal to or slightly lower than that of PAA. On the other hand, the maximum [Ca²⁺]_{bind} of 6CEE-α-CD was approximately 40%. Presumably, both or either of the above-described inclusion of CEE group into the cavity of α-CD (where the CEE group forms a complex with the α-CD) and the small number of CEE per one molecule may reduce the binding capacity. Thus, calcium chelating may be enhanced by the supramolecular structure of the polyrotaxane in relation to increase in the concentration of CEE group.

### Example 8

Trypsin (CE 3.4.21.4.4 type IX, derived from pig spleen), N-α-benzoyl-L-arginine ethylester (BAEE) and N-α-benzoyl-L-arginine (BA) were purchased from Sigma (St. Lois, Mo., U.S.). Other compounds were of the highest-purity.

Trypsin Inhibition Assay

There are two hypotheses which explain the mechanism of trypsin inhibition by polyacrylic acid (PAA): one is calcium ion chelation (Luessen et al., Pharm. Res. 12 (1995) 1293-1298 Luessen et al., Eur. J. Pharm. Sci. 4 (1996) 117-1285; Lussen et al., J. Control. Rel. 45 (1997) 15-23); and the other is its direct interaction with the enzyme (Walker et al., Pharm. Res. 16 (1999) 1074-1080). In order to evaluate the effect that 132CEE-α/E4-PHE-Z may have on the inhibition of trypsin activity, trypsin inhibition assay was performed to examine digestion of N-α-benzoyl-L-arginine ethylester (BAEE) by trypsin in the presence of 132CEE-α/E4-PHE-Z, PAA and 6CEE-α-CD.

The following samples were dissolved in MES/KOH buffer (pH 6.7) for use in trypsin inhibition assay.

a) 0.18% (w/v) PAA

b) 0.75 (w/v) 132CEE-α/E4-PHE-Z

c) 0.66% 6CEE-α-CD

In the assay, 25 mM carboxyl group was used. MES/KOH buffer was used as a control.

N-α-benzoyl-L-arginine ethylester (1.5 mmol) was dissolved in each of the sample solutions. Various dilutions of the substrate solutions (5 ml each) were used in the degradation assay. Degradation experiments were started by adding trypsin (final concentration=24.0 IU/ml) to each sample at 37° C. In order to analyze the degradation using high performance liquid chromatography (HPLC), the reaction solution (50 ul) was sampled at appropriate time points and diluted in 1 ml of phosphoric acid (pH 2) to stop trypsin activity. The degradation product (N-α-benzoyl-L-arginine, BA) was analyzed by the HPLC using a reversed-phase column (COSMOSIL 5C18-AR-II, 250×4.5 mm; Nacalai Tesque, Inc., Kyoto, Japan) at a flow rate of 0.75 ml/min. The mobile phase consisted of: eluate A, 86% (v/v) 10 mM ammonium acetate (pH 4.2) and 14% (v/v) acetonitrile; and eluate B, 80% (v/v) 10 mM ammonium acetate (pH 4.2) and 20% (v/v) acetonitrile. Gradient elution was performed as follows: 0-8 min: 92% A/8% B, isocratic; 8-10 min: 50% A/50% B, linear gradient; 10-13 min: 50% A/50% B, isocratic. BA was detected at 253 nm. Under these conditions, the elution peak of BA was detected at 6.35 min.

The degree of trypsin inhibition was expressed by an inhibition factor (IF) (Madsen et al., Biomaterials 20 (1999) 1701-1708) as follows:

IF=AUC_{control}/AUC_{polymer}

where AUC is the area under BA vs. time curve in the absence (AUC_{control}) or presence (AUC_{polymer}) of polymers.

FIG. 14 shows the effect of conjugates on trypsin activity in the presence or absence of calcium chloride (20 mg/ml). In this experiment, an excess amount of calcium chloride was added just before trypsin reaction to evaluate the effect of calcium ion chelation on trypsin inhibition. In the absence of calcium chloride, the hydrolyzed N-α-benzoyl-L-arginine ethylester (N-α-benzoyl-arginine, BA) increased with time (6CEE-α-CD>>132CEE-α/E4-PHE-Z>PAA). That seems to be an inverse relationship between the amount of hydrolyzed N-α-BA and the calcium chelating ability.

When an excess amount of calcium chloride was added before the degradation, the amount of N-α-benzoyl-arginine (BA) increased over 60 minutes in the presence of 132CEE-α/E4-PHE-Z or PAA when compared with the case without addition of excess calcium chloride, but not in the presence of 6CEE-α-CD. These results suggest that calcium chelation by 132CEE-α/E4-PHE-Z and PAA correlates to trypsin inhibition.

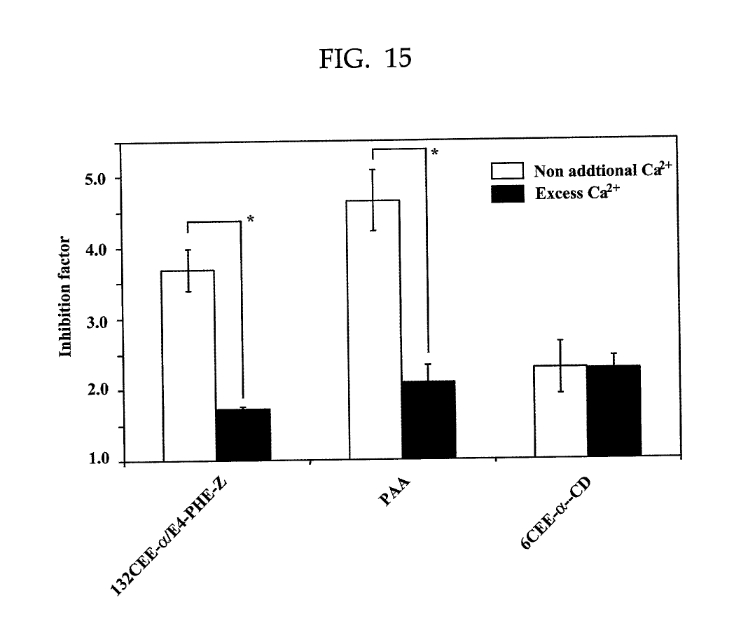

To quantitatively determine the inhibitory effect, inhibition factors (IFs) of the 132CEE-α/E4-PHE-Z, PAA and 6CEE-α-CD during the 60 minute-reaction were calculated (FIG. 15). In FIG. 15, * shows that there is a significant difference in the inhibition factors in t-test (P<0.05). The IF values of 132CEE-α/E4-PHE-Z and PAA significantly decreased by addition of an excess amount of calcium chloride while that of 6CEE-α-CD did not change. Similar results were obtained in a 180 minute-reaction.

In the presence of an excess amount of calcium chloride, 132CEE-α/E4-PHE-Z was suspended in the solution while PAA precipitated in the solution. The precipitation of PAA suggests that all the carboxyl groups in PAA were stoichiometrically involved in calcium chelation (Kriwet, Kissel, 1996) and that the PAA content in the solution decreased. On the other hand, the suspension of 132CEE-α/E4-PHE-Z indicates that there existed CEEs that did not involved in calcium chelation in the solution. This may be supported by the observation that less calcium chelation took place in the 132CEE-α/E4-PHE-Z suspension than in PAA solution (FIG. 13). PAA is considered to bind directly to and thus reduce the activity of trypsin (Walker et al, Pharm. Res. 16 (1999) 1074-1080). Therefore, under the co-presence of an excess amount of calcium chloride and PAA, if all the PAA has been dissolved in the solution in the presence of an excess amount of calcium ion, the IF value will increase. Therefore, the mechanism of trypsin inhibition by 132CEE-α/E4-PHE-Z may be attributed to its relatively weak calcium chelating ability. It can be assumed that the trypsin inhibition by 6CEE-α-CD may be mediated by another mechanism. Presumably, the inhibition mechanism may involve reducing the accessibility of trypsin to N-α-benzoyl-L-arginine ethylester (BAEE) by including the aromatic groups of the N-α-benzoyl-L-arginine ethylester (BAEE) and/or trypsin into the cavity of 6CEE-α-CD (Rekharsky et al., Chem. Rev. 98 (1998) 1875-1917).

The above-described trypsin inhibition experiments showed that trypsin inhibition by carboxyethylester-polyrotaxane was due to calcium chelating rather than to non-specific interaction. Owing to this property, the inventive multivalently interactive molecular assembly can be used as a calcium chelating agent to inhibit, for example, trypsin, or to open the tight junction of small intestine, as well as for other biological effects of calcium chelating.

### Example 9

Inhibition of Trypsin Activity by Various Carboxyethylester-Conjugated Polyrotaxanes Comprising PEG of Different Molecular Weight with Different Number of Threading α-CD

The above Examples showed that the inhibition of enzyme activity by CEE-polyrotaxane may depend on calcium chelation rather than non-specific interaction. In this Example, trypsin inhibition activity was determined using various CEE-polyrotaxanes comprising PEG of different molecular weight with different number of threading α-CDs to examine the inhibition of enzyme activity by the CEE-polyrotaxanes and the calcium-dependency of enzyme activity inhibition.

First, carboxyethylester-polyrotaxanes were synthesized. Here, polyrotaxanes having capping benzyloxy carbonyl-L-tyrosine (Z-L-Tyr) groups at the both ends thereof (MW of PEG: 2000 or 4000) were used. Each of the polyrotaxanes (6.03×10⁻⁶ mol) was stirred heterogeneously in pyridine (solvent) with succinic anhydride (3.26×10⁻⁶ mol). Next, the mixture was precipitated again and washed in a large amount of ether. The resulting precipitate was collected by centrifugation and dried under reduced pressure to give carboxyethylester-polyrotaxane (CEE-polyrotaxane). The amount of α-CDs threaded onto the PEG chain and CEEs introduced were counted by a ¹H-NMR assay. The results are shown in Table 5 below.

TABLE 5
Synthesis of CEE-polyrotaxane

| | | | | # of α | |
|---|---|---|---|---|---|
| | | Mn of | # of CEE/ | -CDs / | % of |
| Sample code^{a} | | PEG | mole^{b} | mole^{b} | threading^{b} |
| 132CEE- | α | 4,000 | 132 | 22 | 49 |
| 22/E4-TYR-Z | | | | | |
| 132CEE- | α | 2,000 | 132 | 22 | 100 |
| 22/E2-TYR-Z | | | | | |
| 96CEE- | α | 2,000 | 96 | 16 | 72 |
| 16/E2-TYR-Z | | | | | |
| 66CEE- | α | 2,000 | 66 | 11 | 50 |
| 11/E2-TYR-Z | | | | | |

^{a}CEE-α/E-TYR-Z: carboxyethylester-polyrotaxane
^{b}Calculate from the ¹H-NMR spectra.

PEGs of MW4,000 and 2,000 were used to synthesize 132CEE-α 22/E4-TYR-Z and 132CEE-α 22/E2-TYR-Z (Table 5) both containing the same number of α-CDs and CEE group, and the ability of these compounds to inhibit trypsin activity was evaluated.

The ability of CEE-polyrotaxanes to inhibit trypsin activity was evaluated as described below.

A model substrate N-α-benzoyl-L-arginine ethylester (BAEE) (1.5 mM) and each CEE-polyrotaxane were dissolved in 2-(N-morpholino) ethane sulfonic acid (MES) buffer (MES/KOH, pH 6.7). Next, the solution was stirred in a thermostat at 37° C. under a constant temperature condition, and added with trypsin (24.0 IU/mL) to start enzymatic degradation. After that, 50 μL of sample was collected at different time points and added to 1 mL of phosphoric acid (pH 2) to quench the reaction. Then, the degraded product N-benzoyl-L-arginine (BA) was quantified by high performance liquid chromatography (HPLC). The amount of carboxyl group of the CEE-polyrotaxane in the solution was kept the same.

HPLC was performed using the following conditions. Column: COSMOSIL 5C18-AR-II (Nacalai Tesque, Inc.); column temperature=37° C.; flow rate=0.75 mL/min; detection by UV (253 nm); developer A=ammonium acetate buffer 86%(v/v)+acetonitrile 14%(v/v), and developer B=ammonium acetate buffer 50%(v/v)+acetonitrile 50%(v/v); gradient=0-8 min A: B=92: 8 (isocratic), 6-18 min A: B=50:50 (linear gradient), 10-13 min A: B=50:50 (isocratic).

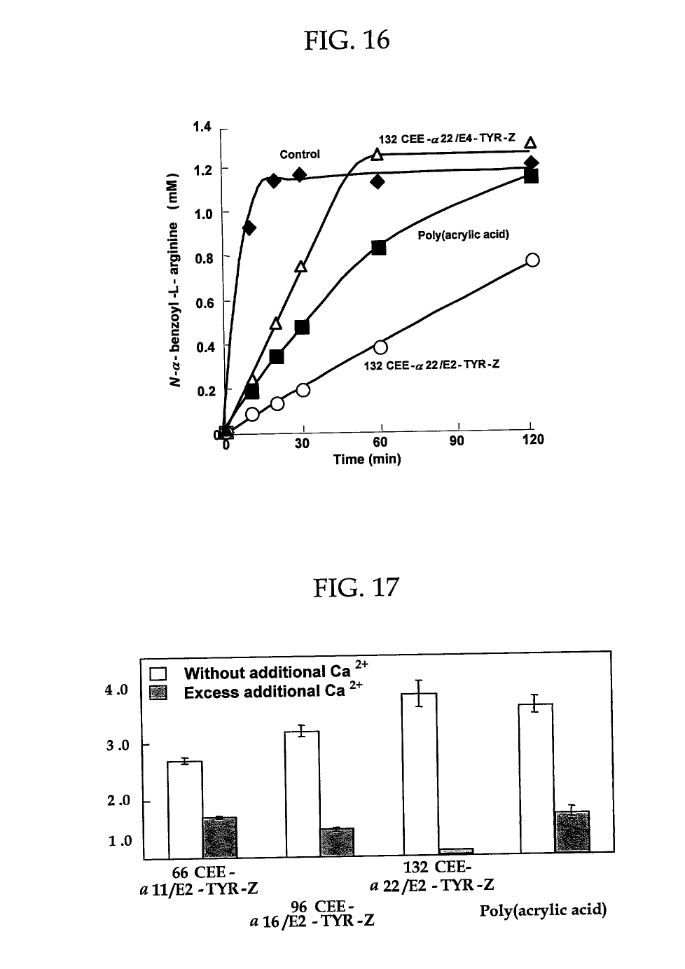

The results are shown in FIG. 16. The ability of 132CEE-α 22/E2-TYR-Z (comprising PEG2,000 as the linear molecule) to inhibit enzyme (trypsin) activity was higher than not only that of 132CEE-α 22/E4-TYR-Z (comprising PEG4,000 as the linear molecule) but also that of PAA, indicating that high carboxyl density due to high α-CD density rather than to the number of CEE may be important for inhibition of trypsin activity.

In order to examine the effect of the number of α-CDs on the inhibition of trypsin activity, CEE-polyrotaxanes comprising PEG-2,000 as the linear molecule with different threading ratio of α-CD (from 100% to 50%) (see Table 5) were used to assay their trypsin inhibition activity. Trypsin inhibition activity is expressed by an IF value.

IF=AUC_{control}/AUC_{polymer}

AUC represents the area under the time vs. BA concentration curve: AUC_{control} the area under the time vs. BA curve obtained using substrate and enzyme alone, and AUC_{polymer} the area under the time vs. BA curve obtained using substrate, enzyme and CEE-polyrotaxane. Greater IF value indicates higher inhibitory effect.

The results are shown in FIG. 17, which shows that higher inhibitory effect can be obtained as the number of CEE and α-CDs increase. Additionally, the effect of addition of an excess amount of calcium was determined to dissect the mechanism of enzyme activity inhibition. The inhibitory effect decreased drastically in 132CEE-α 22/E2-TYR-Z case (with greater threading ratio of α-CD) after addition of an excess amount of calcium, which also suggests that the mechanism of enzyme inhibition by 132CEE-α 22/E2-TYR-Z may depend on calcium chelation.

### Example 10

Evaluation of Multivalent Interaction by Maltose-Polyrotaxane Conjugate

Multivalent interaction was evaluated using maltose-polyrotaxane conjugate.

At first, maltose-polyrotaxane conjugates were synthesized as described below.

Condensation reaction between the carboxyl group of the polyrotaxane in which the hydroxyl groups in α-CDs have been carboxyethylesterified (CEE-PRX) and mono-aminated maltose (β-maltosylamine) was performed using BOP reagent to produce maltose (Mal)-polyrotaxane conjugates. Those were then purified by dialysis. Similarly, Mal-polyacrylic acid (Mal-PAA) was synthesized as the reference sample. The number of both threading α-CD and Mal introduced were determined by ¹H-NMR. Results are shown in Table 6.

TABLE 6
Synthesis of Mal-polyrotaxanes

| | | # of α-CD | | |
|---|---|---|---|---|
| | | (theor. #) | # of | |
| | Mn of | Threading | maltos | Total |
| Sample code^{a} | PEG | percent^{b} | e^{b} | Mn^{b} |
| 88Mal-α 22/E2-TYR | 2,000 | 22 (22) 100% | 88 | 67,000 |
| 120Mal-α 30/E4-TYR | 4,000 | 30 (45) 67% | 120 | 93,000 |
| 340Mal-α 68/E10-TYR | 10,000 | 68 (110) 62% | 340 | 234,000 |
| 510Mal-α 130/E20-TYR | 20,000 | 130 (225) 58% | 510 | 400,000 |
| 830Mal-α 290/E35-TYR | 35,000 | 290 (385) 75% | 830 | 776,000 |
| 1260Mal-α 420/E50-TYR | 50,000 | 420 (550) 78% | 1260 | 995,000 |
| 78-PAA25 | — | — | 78 | 52,000 |

^{a}Mal-α/E-TYR-Z: Maltose-polyrotaxane conjugate
^{b}Calculate from the ¹H-NMR spectra.

Next, hemagglutination inhibition test was performed to evaluate the interaction between Mal-polyrotaxane conjugate and concanavalin A (ConA), then 20 μL of diluted Mal-polyrotaxane conjugate in saline and 20 μL of solution of ConA in saline were dispensed in a 96-well plate (U-bottom), stirred, and then incubated at 37° C. for 30 minutes. Next, 40 μL of 2%(v/v) rat erythrocyte was added to the plate, and the mixture was stirred and then incubated at 37° C. for 30 minutes. The precipitation of erythrocyte was monitored to determine hemagglutination, and the minimum concentration to inhibit hemagglutination was determined. The ConA concentration was set up at a 4-fold higher value than the minimum concentration of ConA at which hemagglutination occurs.

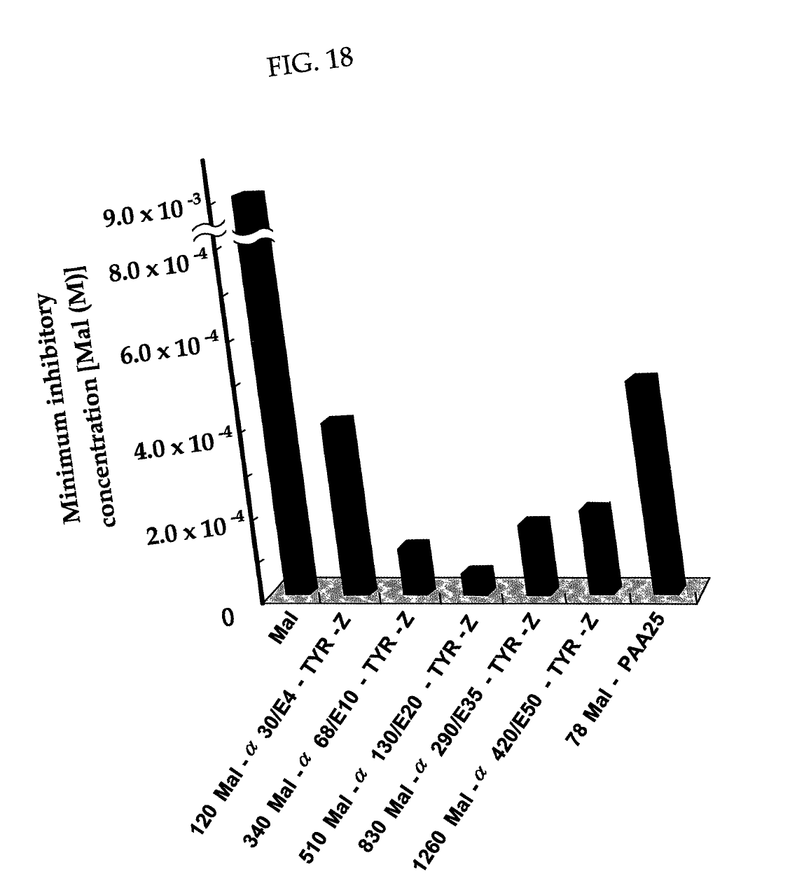

The effect of hemagglutination inhibition by various Mal-polyrotaxane conjugates are shown in FIG. 18. Mal inhibited hemagglutination at 9.1×10⁻³M while Mal-polyrotaxane conjugate from 4.0×10⁻⁴M to 5.1×10⁻⁵M or more. It can be seen from these results that Mal-polyrotaxane conjugate exhibited from 23- to 180-fold higher inhibition than Mal. This result suggests multivalent interaction between Mal and ConA in relation to the polyrotaxane structure. Moreover, 510Mal-α 130/E20-TYR-Z exhibited the highest inhibition, indicating that the supramolecular structure of the polyrotaxane is involved in the multivalent interaction.

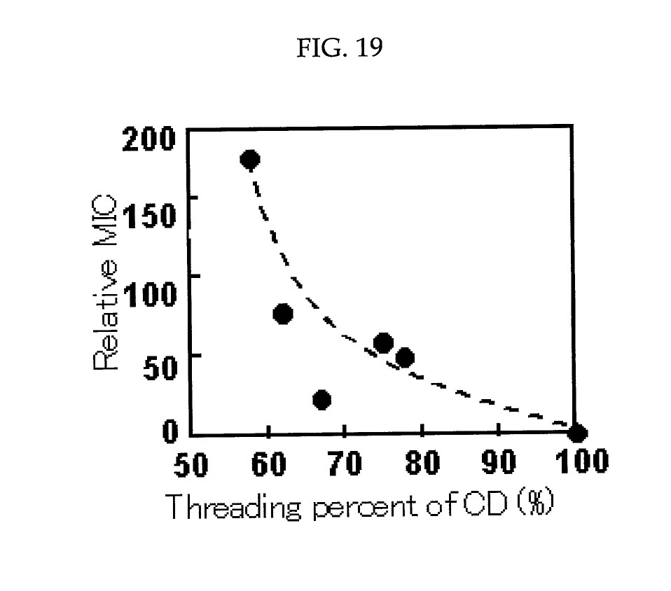

The relationship between the inhibitory effect and the threading ratio of α-CD is shown in FIG. 19. Inhibitory effect was evaluated using the Relative MIC as shown in the following expression.

Relative MIC=(Min. inhibitory conc. of maltose)/(Min inhibitory conc. of maltose in the conjugate)

The results showed that higher relative MIC was obtained with lower threading ratio of α-CD while lower relative MIC with higher threading ratio of α-CD. This is likely to be because, in a polyrotaxane with high threading ratio of α-CD, the high density of α-CDs or Mals causes steric hindrance between ConA and Mal, which may lead to lesser interaction. In a polyrotaxane with smaller number of threading α-CD, individual α-CD may have relatively higher degree of freedom that may cause less steric hindrance, thereby allowing for efficient binding of Mal to the binding sites of ConA.

According to the present invention, a multivalently interactive molecular assembly which can effectively and stably bind to a target substance in vivo or in vitro, a capturing agent comprising said multivalently interactive molecular assembly for capturing an object of interest in vivo or in vitro, a drug carrier that aids administration of a drug, a calcium chelating agent that can effectively chelate calcium, and a drug enhancer that can be administered with a drug to assist in, for example, absorption of the drug can be provided.

What is claimed is:

1. A multivalently interactive molecular assembly comprising a plurality of at least one of functional groups and ligands,
wherein a ratio between Rₕ and R_{g} which is expressed by Rₕ/R_{g} is 1.0 or less;
(where Rₕ is a hydrodynamic radius calculated from dynamic light scattering (DLS) assay performed in aqueous solution; and R_{g} is a radius of gyration determined based on the Zimm plot generated using data obtained by static light scattering (SLS) assay).

2. A multivalently interactive molecular assembly comprising a plurality of at least one of functional groups and ligands,
wherein a diffusion constant D calculated from a dynamic light scattering assay performed in aqueous solution increases as scattering vector constant K increases.

3. A multivalently interactive molecular assembly comprising:
a plurality of cyclic molecules;
a linear molecule which threads through the cyclic molecules to hold the cyclic molecules together; and
bulky substituents capping both ends of the linear molecule;
wherein at least two of the plurality of cyclic molecules are substituted with at least one of a functional group and a ligand.

4. A multivalently interactive molecular assembly according to claim 3, wherein the bulky substituents are linked to the linear molecule in a state to allow degrading of the bulky substituents in vivo.

5. A multivalently interactive molecular assembly according to claim 3, wherein the multivalently interactive molecular assembly is a polyrotaxane.

6. A multivalently interactive molecular assembly according to claim 3, wherein the cyclic molecules are cyclodextrin.

7. A multivalently interactive molecular assembly according to claim 6, wherein a glucose C3 proton and a glucose C5 proton each present in a cavity of the cyclodextrin exhibits a shift in one of an upfield and a downfield of 0.1 to 1.0 ppm, when compared to a glucose C3 proton and a glucose C5 proton each present in a cavity of free cyclodextrin as determined by one-dimensional ¹H-NMR spectroscopy.

8. A multivalently interactive molecular assembly according to claim 6, wherein the linear molecule threading through the cyclodextrins exhibits a shift in one of an upfield and a downfield of 0.01 to 1.0 ppm when compared to the linear molecule which is not threading through the cyclodextrins as determined by one-dimensional ¹H-NMR spectroscopy.

9. A multivalently interactive molecular assembly according to claim 6, wherein a cross peak caused by a nuclear Overhauzer effect working in between glucose C3 and C5 protons present in a cavity of the cyclodextrin and protons present in the linear molecule is observed as determined by a two-dimensional ¹H-NMR spectrum, and values of chemical shifts are 3.0 to 4.0 ppm for the glucose C3 and C5 protons, and 1.0 to 6.0 ppm for the linear molecule.

10. A multivalently interactive molecular assembly according to claim 6, wherein no melting peak is detected for the linear molecule in the DSC chart of differential scanning calorimetry.

11. A multivalently interactive molecular assembly according to claim 3, wherein the functional group contains a caboxyl group at an end thereof.

12. A multivalently interactive molecular assembly according to claim 12, wherein the functional group containing a caboxyl group at an end thereof is a carboxyalkoxycarbonyl group.

13. A multivalently interactive molecular assembly according to claim 3, wherein the ligand is a sugar ligand.

14. A multivalently interactive molecular assembly comprising:
a plurality of cyclodextrin molecules;
a linear molecule which threads through the plurality of cyclodextrin molecules to hold the plurality of cyclodextrin molecules together; and
bulky substituents capping both ends of the linear molecule;
wherein a peak area of C6 primary hydroxyl group, C2 secondary hydroxyl group and C3 secondary hydroxyl group in at least two of the cyclodextrin molecules are reduced by 10 to 95% than a peak area of the corresponding hydroxyl group in a cyclodextrin with no substituents, as determined by a two-dimensional ¹H-NMR spectroscopy.

15. A capturing agent comprising at least a multivalently interactive molecular assembly which can capture an object of interest,
wherein the multivalently interactive molecular assembly comprises:
a plurality of cyclic molecules;
a linear molecule which threads through the cyclic molecules to hold the cyclic molecules together; and
bulky substituents capping both ends of the linear molecule;
wherein at least two of the plurality of cyclic molecules are substituted with one of a functional group and a ligand.

16. A drug carrier which can be bonded to a drug comprising at least a multivalently interactive molecular assembly,
wherein the multivalently interactive molecular assembly comprises:
a plurality of cyclic molecules;
a linear molecule which threads through the cyclic molecules to hold the cyclic molecules together; and
bulky substituents capping both ends of the linear molecule;
wherein at least two of the plurality of cyclic molecules are substituted with one of a functional group and a ligand.

17. A calcium chelating agent which can chelate calcium comprising at least a multivalently interactive molecular assembly,
wherein the multivalently interactive molecular assembly comprises:
a plurality of cyclic molecules;
a linear molecule which threads through the cyclic molecules to hold the cyclic molecules together; and
bulky substituents capping both ends of the linear molecule;
wherein at least two of the plurality of cyclic molecules are substituted with a functional group containing caboxyl group at an end thereof.

18. A drug enhancer for enhancing efficacy of the drug comprising at least a multivalently interactive molecular assembly,
wherein the multivalently interactive molecular assembly comprises:
a plurality of cyclic molecules;
a linear molecule which threads through the cyclic molecules to hold the cyclic molecules together; and
bulky substituents capping both ends of the linear molecule;
wherein at least two of the plurality of cyclic molecules are substituted with a functional group containing caboxyl group at an end thereof.

19. A drug enhancer according to claim 18, wherein the at least two of the plurality of cyclic molecules are multivalently interactive molecular assembly substituted with a ligand.

20. Polyrotaxane which can be used in a multivalently interactive molecular assembly,
wherein the multivalently interactive molecular assembly comprises:
a plurality of cyclic molecules;
a linear molecule which threads through the cyclic molecules to hold the cyclic molecules together; and
bulky substituents capping both ends of the linear molecule;
wherein at least two of the plurality of cyclic molecules are substituted with one of functional groups and ligands.
